(19) United States
(12) Patent Application Publication
(10) Pub. No.: US 2010/0261259 A1
(43) Pub. Date: Oct. 14, 2010

(54) Expression of Genes from Gram Negative Bacteria in Fungi

(75) Inventors: Tomoko Matsui, Chiba-shi (JP); Pia Francke Johannesen, Skovlunde (DK); Shinobu Takagi, Funabashi (JP); Thomas Agersten Poulsen, Ballerup (DK); Noriko Tsutsumi, Ichikawa-shi (JP); Zheng Liu, Beijing (CN); XiangYu Kong, Beijing (CN)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/376,372

(22) PCT Filed: Aug. 3, 2007
(86) PCT No.: PCT/EP2007/058077

§ 371 Date: Feb. 4, 2009

Related U.S. Application Data
(60) Provisional application No. 60/842,722, filed on Sep. 7, 2006.

(30) Foreign Application Priority Data
Aug. 8, 2006 (DK) PA 2006 01042

(51) Int. Cl. C12N 1/19; C12N 1/15; C07H 21/04

(52) U.S. Cl. 435/254.3; 435/254.11; 435/254.2; 435/254.23; 435/254.5; 435/254.6; 435/254.7; 435/254.8; 536/23.2

(57) ABSTRACT

The present invention provides a method for the recombinant expression of polypeptides originating from gram negative bacteria, in a fungal host suitable for industrial production. In a first aspect the present invention relates to a method for recombinant expression of a polypeptide from a gram negative bacterium in a fungal host cell, comprising the steps: i) providing a nucleic acid sequence encoding the polypeptide, said nucleic acid sequence comprising a first nucleic acid sequence encoding a fungal signal peptide and a second nucleic acid sequence encoding the polypeptide, having at least one modified codon, wherein the modification does not change the amino acid encoded by said codon and the nucleic acid sequence of said codon is different compared to the corresponding codon in the wild type nucleic acid sequence present in the said gram negative bacterium; ii) expressing the modified nucleic acid sequence in the fungal host.

### REFERENCE TO A SEQUENCE LISTING

This application contains a Sequence Listing in computer readable form. The computer readable form is incorporated herein by reference.

### FIELD OF THE INVENTION

The present invention relates to methods for recombinant expression of polypeptides originating from gram negative bacteria in a fungal host organism as well as to modified nucleic acid sequences encoding such polypeptides. Particularly the polypeptide is a phytase.

### BACKGROUND OF THE INVENTION

Recombinant expression of polypeptides originating from gram negative bacteria in a fungal host is not always straight forward and obtaining sufficient yields is hard to predict for a given protein in a given expression host organism. Several differences exist between expression in a gram negative bacterium and a fungus. In gram negative bacteria genes do not comprise introns and codon usage is different from fungal codon usage. For secreted proteins, the secretion machinery is believed to be another limitation as there are huge differences between the secretion machinery of gram negative bacteria and eukaryotic cells like filamentous fungi. Attempts to modify a given sequence of a poorly expressed gene might furthermore result in the introduction of undesired changes such as the introduction of cryptic introns as described in WO 97/49821. In order to successfully express a gene sequence originating from a gram negative bacterium in a fungus therefore requires modification of a lot of parameters which combined may result in a sufficient expression in the fungal host cell.

### SUMMARY OF THE INVENTION

The present invention provides a method for the recombinant expression of polypeptides originating from gram negative bacteria, in a fungal host suitable for industrial production.

In a first aspect the present invention relates to a method for recombinant expression of a polypeptide from a gram negative bacterium in a fungal host cell, comprising the steps: i) providing a nucleic acid sequence encoding the polypeptide, said nucleic acid sequence comprising a first nucleic acid sequence encoding a fungal signal peptide and a second nucleic acid sequence encoding the polypeptide, having at least one modified codon, wherein the modification does not change the amino acid encoded by said codon and the nucleic acid sequence of said codon is different compared to the corresponding codon in the wild type nucleic acid sequence present in the said gram negative bacterium; ii) expressing the modified nucleic acid sequence in the fungal host.

In a second aspect the present invention relates to a host cell comprising a DNA construct, said DNA construct comprising: i) a first nucleic acid sequence encoding a fungal signal peptide; ii) a second nucleic acid sequence encoding a polypeptide from a gram negative bacterium; and wherein the second nucleic acid sequence comprises at least one modified codon compared to the wild type gene, which modification does not change the amino acid encoded by said codon.

In a third aspect the present invention relates to modified nucleic acid sequences encoding a phytase polypeptide and capable of expression in a fungal host organism, wherein said modified nucleic acid sequences differ in at least one codon from each wild type nucleic acid sequence encoding said phytase polypeptide.

### DEFINITIONS

Phytase: In the present context a phytase is an enzyme which catalyzes the hydrolysis of phytate (myo-inositol hexakisphosphate) to (1) myo-inositol and/or (2) mono-, di-, tri-, tetra- and/or penta-phosphates thereof and (3) inorganic phosphate. Three different types of phytases are known: A so-called 3-phytase (alternative name 1-phytase; a myo-inositol hexaphosphate 3-phosphohydrolase, EC 3.1.3.8), a so-called 4-phytase (alternative name 6-phytase, name based on 1 L-numbering system and not 1 D-numbering, EC 3.1.3.26), and a so-called 5-phytase (EC 3.1.3.72). Phytases belonging to the classes EC 3.1.3.8 and EC 3.1.3.26 have both been found in gram negative bacteria.

For the purposes of the present invention phytase activity may be, preferably is, deter-mined in the unit of FYT, one FYT being the amount of enzyme that liberates 1 micro-mol inorganic ortho-phosphate per min. under the following conditions: pH 5.5; temperature 37° C.; substrate: sodium phytate (C₆H₆O₂₄P₆Na₁₂) in a concentration of 0.0050 mol/l. Suitable phytase assays are described in Example 1 of WO 00/20569. FTU is for determining phytase activity in feed and premix. A plate assay is described in the examples below. Preferred examples of phytases are bacterial phytases, e.g. derived from the following:

i. Escherichia coli (e.g. U.S. Pat. No. 6,110,719);
ii. Citrobacter, such as Citrobacter freundii (disclosed in WO 2006/038062, WO 2006/038128, or with the sequence of UniProt Q676V7), Citrobacter braakii (disclosed in WO 2004/085638 (Geneseqp ADU50737), and WO 2006/037328), and Citrobacter amalonaticus or Citrobacter gillenii (disclosed in WO 2006/037327);
iii. Other bacterial phytases such as the phytase from Buttiauxella (disclosed in WO 2006/043178).

Isolated polypeptide: The term “isolated polypeptide” as used herein refers to a poly-peptide which is at least 20% pure, preferably at least 40% pure, more preferably at least 60% pure, even more preferably at least 80% pure, most preferably at least 90% pure, and even most preferably at least 95% pure, as determined by SDS-PAGE.

Substantially pure polypeptide: The term “substantially pure polypeptide” denotes herein a polypeptide preparation which contains at most 10%, preferably at most 8%, more preferably at most 6%, more preferably at most 5%, more preferably at most 4%, at most 3%, even more preferably at most 2%, most preferably at most 1%, and even most preferably at most 0.5% by weight of other polypeptide material with which it is natively associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation.

The polypeptides of the present invention are preferably in a substantially pure form. In particular, it is preferred that the polypeptides are in “essentially pure form”, i.e., that the poly-peptide preparation is essentially free of other polypeptide material with which it is natively associated. This can be accomplished, for example, by preparing the polypeptide by means of well-known recombinant methods or by classical purification methods.

Herein, the term “substantially pure polypeptide” is synonymous with the terms “isolated polypeptide” and “polypeptide in isolated form.”

Identity: For purposes of the present invention, the degree of identity between two nucleotide sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

Subsequence: The term “subsequence” is defined herein as a nucleotide sequence having one or more nucleotides deleted from the 5′ and/or 3′ end or a homologous sequence thereof, wherein the subsequence encodes a polypeptide fragment having phytase activity.

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Substantially pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form. In particular, it is preferred that the polynucleotides disclosed herein are in “essentially pure form”, i.e., that the polynucleotide preparation is essentially free of other polynucleotide material with which it is natively associated. Herein, the term “substantially pure polynucleotide” is synonymous with the terms “isolated polynucleotide” and “polynucleotide in isolated form.” The polynucleotides may be of genomic, cDNA, RNA, semisynthetic, synthetic origin, or any combinations thereof.

cDNA: The term “cDNA” is defined herein as a DNA molecule which can be prepared by reverse transcription from a mature, spliced, mRNA molecule obtained from a eukaryotic cell. cDNA lacks intron sequences that are usually present in the corresponding genomic DNA. The initial, primary RNA transcript is a precursor to mRNA which is processed through a series of steps before appearing as mature spliced mRNA. These steps include the removal of intron sequences by a process called splicing. cDNA derived from mRNA lacks, therefore, any intron sequences.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

Control sequence: The term “control sequences” is defined herein to include all components, which are necessary or advantageous for the expression of a polynucleotide encoding a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be pro-vided with linkers for the purpose of introducing specific restriction sites facilitating ligation of the control sequences with the coding region of the nucleotide sequence encoding a polypeptide.

Operably linked: The term “operably linked” denotes herein a configuration in which a control sequence is placed at an appropriate position relative to the coding sequence of the polynucleotide sequence such that the control sequence directs the expression of the coding sequence of a polypeptide.

Coding sequence: When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG. The coding sequence may be a DNA, cDNA, or recombinant nucleotide sequence.

Expression: The term “expression” includes any step involved in the production of the polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide of the invention, and which is operably linked to additional nucleotides that provide for its expression.

Host cell: The term “host cell”, as used herein, includes any cell type which is susceptible to transformation, transfection, transduction, and the like with a nucleic acid construct comprising a polynucleotide of the present invention.

Modification: The term “modification” means herein any chemical modification of or genetic manipulation of the DNA encoding the polypeptide from a gram negative bacterium. The modification(s) can be substitution(s), deletion(s) and/or insertions(s) of the amino acid(s) as well as replacement(s) of amino acid side chain(s).

Synthetic variant: When used herein, the term “synthetic variant” means a modified nucleotide sequence, wherein the modified nucleotide sequence is obtained through human intervention by modification of the “wild type” nucleotide sequence encoding the wild type poly-peptide.

Wild type nucleotide sequence: The term “wild type nucleotide sequence” as used herein refers to any natural variant of a polynucleotide originating from a gram negative bacterium as opposed to the modified nucleotide sequence or synthetic variant according to the invention in which modifications have been introduced into the nucleotide sequence in order to improve expression in the fungal host.

### DETAILED DESCRIPTION OF THE INVENTION

In general expression of a secreted and correctly processed polypeptide in a fungus involves a number of steps any of which could be a limiting step.

First the inserted gene encoding a polypeptide from a gram negative bacterium is transcribed to hnRNA. Then the hnRNA is transported from the nucleus to the cytosol, and during this process it is maturated to mRNA. Generally a mRNA pool is established in the cytosol in order to sustain translation. The mRNA is then translated to a protein precursor, and this precursor is subsequently secreted to the endoplasmatic reticulum (ER) either co-translationally or post-translationally. Upon translocation into the ER the secretion signal peptide is cleaved of by a signal peptidase, and the resulting protein is folded in the ER. Secretion of the protein to the golgi apparatus follows when proper folding has been recognized by the cell. Here the propeptide will be cleaved to release the mature polypeptide. Thus numerous possibilities exist for preventing sufficient expression of a gene sequence in a given host organism.

In order to provide efficient expression of a polynucleotide sequence encoding a desired protein the translation process has to be efficient. One object of the present invention is therefore to optimize the mRNA sequence encoding the polypeptide from a gram negative bacterium in order to obtain sufficient expression in a fungal host cell.

In one embodiment the present invention relates to a method for recombinant expression of a polypeptide in a fungal host organism comprising modifying a wild type nucleic acid sequence to provide a synthetic variant encoding the same polypeptide which can be expressed in the fungal host cell of choice.

The modified nucleic acid sequence may be obtained by a) providing a wild type nucleic acid sequence encoding a polypeptide and b) modifying at least one codon of said nucleic acid sequence so that the modified nucleic acid sequence differs in at least one codon from each wild type nucleic acid sequence encoding said polypeptide. Methods for modifying nucleic acid sequences are well known to a person skilled in the art. In particular said modification does not change the identity of the amino acid encoded by said nucleic acid sequence.

Thus in one aspect the object of the present invention is provided by a method for recombinant expression of a polypeptide from a gram negative bacterium in a fungal host cell, comprising the steps:

i) providing a nucleic acid sequence encoding the polypeptide, said nucleic acid sequence comprising a first nucleic acid sequence encoding a fungal signal peptide and a second nucleic acid sequence encoding the polypeptide, having at least one modified codon, wherein the modification does not change the amino acid encoded by said codon and the nucleic acid sequence of said codon is different compared to the corresponding codon in the wild type nucleic acid sequence present in the said gram negative bacterium; and
ii) expressing the modified nucleic acid sequence in the filamentous fungal host.

The starting nucleic acid sequence to be modified according to this embodiment is a naturally occurring or wild type nucleic acid sequence encoding the polypeptide of interest or any nucleic acid sequence encoding the polypeptide which cannot be sufficiently expressed in a fungal host.

Modifications according to the invention, comprises any modification of the base triplet and in a particular embodiment they comprise any modification which does not change the identity of the amino acid encoded by said codon, i.e. the amino acid encoded by the original codon and the modified codon is the same. In most cases the modification will be at the third position, however, in a few cases the modification may also be at the first or the second position. How to modify a codon also without modifying the resulting amino acid is known to the skilled person.

The number of codons which should differ or the number of modifications needed in order to obtain sufficient expression may vary. Thus according to a further embodiment of the invention the modified nucleic acid sequence differs in at least 2 codons from each wild type nucleic acid sequence encoding said polypeptide or at least 3 codons have been modified, particularly at least 4 codons, more particularly at least 5 codons, more particularly at least 10 codons, more particularly at least 15 codons, even more particularly at least 25 codons.

It has furthermore been found, that by changing the codon usage of the wild type nucleic acid sequence to be selected among the codons preferably used by the fungus used as a host, the expression of polypeptides from gram negative bacteria is now possible. Such codons are said to be “optimized” for expression.

Due to the degeneracy of the genetic code and the preference of certain preferred codons in particular organisms/cells the expression level of a protein in a given host cell can in some instances be improved by optimizing the codon usage. In the present case the yields of different phytases were increased dramatically when wild type nucleic acid sequences encoding such phytases were optimized by, among other things, codon optimization and expressed in Aspergillus or Pichia.

In the present invention “codon optimized” means that due to the degeneracy of the genetic code more than one triplet codon can be used for each amino acid. Some codons will be preferred in a particular organism and by changing the codon usage in a wild type gene to a codon usage preferred in a particular expression host organism the codons are said to be optimized. Codon optimization can be performed e.g. as described in Gustafsson et al., 2004, (Trends in Biotechnology vol. 22 (7); Codon bias and heterologous protein expression), and U.S. Pat. No. 6,818,752.

Codon optimization may be based on the average codon usage for the host organism or it can be based on the codon usage for a particular gene which is know to be expressed in high amounts in a particular host cell.

In one embodiment of the invention the wild type polypeptide is encoded by a modified nucleic acid sequence codon optimized in at least 10% of the codons, more particularly at least 20%, or at least 30%, or at least 40%, or particularly at least 50%, more particularly at least 60%, more particularly at least 75%, and most particularly at least 90%. Thus the modified nucleic acid sequence may differ in at least 10% of the codons from each wild type nucleic acid sequence encoding said wild type serum albumin polypeptide, more particularly in at least 20%, or in at least 30%, or in at least 40%, or particularly in at least 50%, more particularly in at least 60%, more particularly in at least 75%, and most particularly at least 90%. In particular said codons may differ because they have been codon optimized as compared with a wild type nucleic acid sequence encoding a wild type polypeptide.

Particularly 100% of the nucleic acid sequence has been codon optimized to match the preferred codons used in fungi.

The codon optimization corresponding to a particular host cell can in a further embodiment be based on a general codon usage in that particular host cell or it can be based on the codon usage of a particular gene. Particularly the said gene is a highly expressed gene.

In one embodiment the codon usage of the at least one modified codon corresponds to the codon usage of a fungal host cell selected from the group consisting of Acremonium, Aspergillus, Fusarium, Humicola, Mucor, Myceliophthora, Neurospora, Penicillium, Thielavia, Tolypocladium, Trichoderma or Pichia.

In a further embodiment the codon usage corresponds to the codon usage of Aspergillus awamori, Aspergillus foetidus, Aspergillus japonicus, Aspergillus niger, Aspergillus nidulans, or Aspergillus oryzae.

In another particular embodiment the codon usage corresponds to the codon usage in Pichia. Particularly the codon usage of Pichia pastoris.

In a particular embodiment the codon optimization corresponds to the codon usage of alpha amylase from Aspergillus oryzae, also known as Fungamyl™ (WO 2005/019443), which is a protein known to be expressed in high levels in filamentous fungi.

In the present context an expression level corresponding to at least 20% of the total amount of secreted protein constitutes the protein of interest is considered a high level of expression. Particularly at least 30%, more particularly at least 40%, even more particularly at least 40%, most particularly at least 50%.

In practice the optimization according to the invention comprises the steps:

i) the nucleic acid sequence encoding the polypeptide is codon optimized as explained in more detail below;
ii) the resulting modified sequence is checked for a balanced GC-content (approximately 45-55%); and
iii) the resulting modified sequence from step ii) is checked or edited further as explained below.

### Codon Optimization Protocol:

The codon usage of a single gene, a number of genes or a whole genome can be calculated with the program cusp from the EMBOSS-package (http://www.emboss.org).

The starting point for the optimization is the amino acid sequence of the protein or a nucleic acid sequence coding for the protein together with a codon-table. By a codon-optimized gene, we understand a nucleic acid sequence, encoding a given protein sequence and with the codon statistics given by a codon table.

The codon statistics referred to is a column in the codon-table called “Fract” in the output from cusp-program and which describes the fraction of a given codon among the other synonymous codons. We call this the local score. If for instance 80% of the codons coding for F is TTC and 20% of the codons coding for F are TTT, then the codon TTC has a local score of 0.8 and TTT has a local score of 0.2.

The codons in the codon table are re-ordered first by encoding amino acid (e.g. alphabetically) and then increasingly by the score. In the example above, ordering the codons for F as TTT, TTC. Cumulated scores for the codons are then generated by adding the scores in order. In the example above TTT has a cumulated score of 0.2 and TTC has a cumulated score of 1. The most used codon will always have a cumulated score of 1.

In order to generate a codon optimized gene the following is performed. For each position in the amino acid sequence, a random number between 0 and 1 is generated. This is done by the random-number generator on the computer system on which the program runs. The first codon is chosen as the codon with a cumulated score greater than or equal to the generated random number. If, in the example above, a particular position in the gene is “F” and the random number generator gives 0.5, TTC is chosen as codon.

The following strategy was used to make sure that the designed synthetic genes would not be spliced in the expression host.

First consensus branch-point motifs (CT[AG]A[CT]) were removed, by locally redesigning the sequence, after the same method as the full sequence was designed.

Then a number of designed genes were run through the NetGene2 splice-site prediction program (REF). For expression in Aspergillus oryzae, the “Aspergillus”-intron model was used, and for Pichia pastoris, the “Yeast” intron model was used. Only genes, that did not have predicted donor-sites in front of any predicted acceptor sites were selected. The NetGene2 program can be accessed through the public server: http://www.cbs.dtu.dk/services/NetGene2/ and is also described in: S. M. Hebsgaard, P. G. Korning, N. Tolstrup, J. Engelbrecht, P. Rouze, S. Brunak: Splice site prediction in Arabidopsis thaliana DNA by combining local and global sequence information, Nucleic Acids Research, 1996, Vol. 24, No. 17, 3439-3452.

Codon tables showing the codon usage of the alpha amylase from Aspergillus oryzae and a codon table which can be used for a gene to be expressed in Pichia are given below.

TABLE 1
Codon usage for the A. oryzae alpha amylase.
(CUSP codon usage file)

| Codon | Amino acid | Fract |
|---|---|---|
| GCA | A | 0.286 |
| GCC | A | 0.357 |
| GCG | A | 0.238 |
| GCT | A | 0.119 |
| TGC | C | 0.222 |
| TGT | C | 0.778 |
| GAC | D | 0.524 |
| GAT | D | 0.476 |
| GAA | E | 0.417 |
| GAG | E | 0.583 |
| TTC | F | 0.800 |
| TTT | F | 0.200 |
| GGA | G | 0.233 |
| GGC | G | 0.419 |
| GGG | G | 0.116 |
| GGT | G | 0.233 |
| CAC | H | 0.571 |
| CAT | H | 0.429 |
| ATA | I | 0.071 |
| ATC | I | 0.679 |
| ATT | I | 0.250 |
| AAA | K | 0.350 |
| AAG | K | 0.650 |
| CTA | L | 0.081 |
| CTC | L | 0.351 |
| CTG | L | 0.162 |
| CTT | L | 0.108 |
| TTA | L | 0.027 |
| TTG | L | 0.270 |
| ATG | M | 1.000 |
| AAC | N | 0.885 |
| AAT | N | 0.115 |
| CCA | P | 0.136 |
| CCC | P | 0.364 |
| CCG | P | 0.227 |
| CCT | P | 0.273 |
| CAA | Q | 0.250 |
| CAG | Q | 0.750 |
| AGA | R | 0.000 |
| AGG | R | 0.300 |
| CGA | R | 0.200 |
| CGC | R | 0.200 |
| CGG | R | 0.200 |
| CGT | R | 0.100 |
| AGC | S | 0.162 |
| AGT | S | 0.108 |
| TCA | S | 0.108 |
| TCC | S | 0.243 |
| TCG | S | 0.270 |
| TCT | S | 0.108 |
| ACA | T | 0.250 |
| ACC | T | 0.325 |
| ACG | T | 0.200 |
| ACT | T | 0.225 |
| GTA | V | 0.129 |
| GTC | V | 0.387 |
| GTG | V | 0.323 |
| GTT | V | 0.161 |
| TGG | W | 1.000 |
| TAC | Y | 0.686 |
| TAT | Y | 0.314 |
| TAA | * | 1.000 |
| TAG | * | 0.000 |
| TGA | * | 0.000 |

TABLE 2
Codon usage for Pichia.

| Codon | Amino acid | Fract |
|---|---|---|
| GCA | A | 0.230 |
| GCC | A | 0.260 |
| GCG | A | 0.060 |
| GCT | A | 0.450 |
| TGC | C | 1.000 |
| TGT | C | 0.000 |
| GAC | D | 0.500 |
| GAT | D | 0.500 |
| GAA | E | 0.500 |
| GAG | E | 0.500 |
| TTC | F | 0.500 |
| TTT | F | 0.500 |
| GGA | G | 0.333 |
| GGC | G | 0.333 |
| GGG | G | 0.000 |
| GGT | G | 0.333 |
| CAC | H | 1.000 |
| CAT | H | 0.000 |
| ATA | I | 0.000 |
| ATC | I | 0.390 |
| ATT | I | 0.610 |
| AAA | K | 0.450 |
| AAG | K | 0.550 |
| CTA | L | 0.000 |
| CTC | L | 0.110 |
| CTG | L | 0.219 |
| CTT | L | 0.219 |
| TTA | L | 0.000 |
| TTG | L | 0.452 |
| ATG | M | 1.000 |
| AAC | N | 0.520 |
| AAT | N | 0.480 |
| CCA | P | 0.631 |
| CCC | P | 0.231 |
| CCG | P | 0.138 |
| CCT | P | 0.000 |
| CAA | Q | 0.500 |
| CAG | Q | 0.500 |
| AGA | R | 0.533 |
| AGG | R | 10.167 |
| CGA | R | 0.122 |
| CGC | R | 0.000 |
| CGG | R | 0.000 |
| CGT | R | 0.178 |
| AGC | S | 0.136 |
| AGT | S | 0.000 |
| TCA | S | 0.000 |
| TCC | S | 0.303 |
| TCG | S | 0.121 |
| TCT | S | 0.439 |
| ACA | T | 0.000 |
| ACC | T | 0.329 |
| ACG | T | 0.145 |
| ACT | T | 0.526 |
| GTA | V | 0.000 |
| GTC | V | 0.282 |
| GTG | V | 0.224 |
| GTT | V | 0.494 |
| TGG | W | 1.000 |
| TAC | Y | 1.000 |
| TAT | Y | 0.000 |
| TGA | * | 0.000 |
| TAG | * | 0.000 |
| TAA | * | 1.000 |

### Introns

Eukaryotic genes may be interrupted by intervening sequences (introns) which must be modified in precursor transcripts in order to produce functional mRNAs. This process of intron removal is known as pre-mRNA splicing. Usually, a branchpoint sequence of an intron is necessary for intron splicing through the formation of a lariat. Signals for splicing reside directly at the boundaries of the intron splice sites. The boundaries of intron splice sites usually have the consensus intron sequences GT and AG at their 5′ and 3′ extremities, respectively. While no 3′ splice sites other than AG have been reported, there are reports of a few exceptions to the 5′ GT splice site. For example, there are precedents where CT or GC is substituted for GT at the 5′ boundary. There is also a strong preference for the nucleotide bases ANGT to follow GT where N is A, C, G, or T (primarily A or T in Saccharomyces species), but there is no marked preference for any particular nucleotides to precede the GT splice site. The 3′ splice site AG is primarily preceded by a pyrimidine nucleotide base (Py), i.e., C or T.

The number of introns that can interrupt a fungal gene ranges from one to twelve or more introns (Rymond and Rosbash, 1992, In, E. W. Jones, J. R. Pringle, and J. R. Broach, editors, The Molecular and Cellular Biology of the Yeast Saccharomyces, pages 143-192, Cold Spring Harbor Laboratory Press, Plainview, N.Y.; Gurr et al., 1987, In Kinghorn, J. R. (ed.), Gene Structure in Eukaryotic Microbes, pages 93-139, IRL Press, Oxford). They may be distributed throughout a gene or situated towards the 5′ or 3′ end of a gene. In Saccharomyces cerevisiae, introns are located primarily at the 5′ end of the gene. Introns may be generally less than 1 kb in size, and usually are less than 400 by in size in yeast and less than 100 by in filamentous fungi.

The Saccharomyces cerevisiae intron branchpoint sequence 5′-TACTAAC-3′ rarely appears exactly in filamentous fungal introns (Gurr et al., 1987, supra). Sequence stretches closely or loosely resembling TACTAAC are seen at equivalent points in filamentous fungal introns with a general consensus NRCTRAC where N is A, C, G, or T, and R is A or G. For ex-ample, the fourth position T is invariant in both the Neurospora crassa and Aspergillus nidulans putative consensus sequences. Furthermore, nucleotides G, A, and C predominate in over 80% of the positions 3, 6, and 7, respectively, although position 7 in Aspergillus nidulans is more flexible with only 65% C. However, positions 1, 2, 5, and 8 are much less strict in both Neurospora crassa and Aspergillus nidulans. Other filamentous fungi have similar branchpoint stretches at equivalent positions in their introns, but the sampling is too small to discern any definite trends.

The heterologous expression of a gene encoding a polypeptide in a fungal host strain may result in the host strain incorrectly recognizing a region within the coding sequence of the gene as an intervening sequence or intron. For example, it has been found that intron— containing genes of filamentous fungi are incorrectly spliced in Saccharomyces cerevisiae (Gurr et al., 1987, In Kinghorn, J. R. (ed.), Gene Structure in Eukaryotic Microbes, pages 93-139, IRL Press, Oxford). Since the region is not recognized as an intron by the parent strain from which the gene was obtained, the intron is called a cryptic intron. This improper recognition of an intron, referred to herein as a cryptic intron, may lead to aberrant splicing of the precursor mRNA molecules resulting in no production of biologically active polypeptide or in the production of several populations of polypeptide products with varying biological activity.

“Cryptic intron” is defined herein as a region of a coding sequence that is incorrectly recognized as an intron which is excised from the primary mRNA transcript. A cryptic intron preferably has 10 to 1500 nucleotides, more preferably 20 to 1000 nucleotides, even more preferably 30 to 300 nucleotides, and most preferably 30 to 100 nucleotides.

The presence of cryptic introns can in particular be a problem when trying to express proteins in organisms which have a less strict requirement to what sequences are necessary in order to define an intron. Such “sloppy” recognition can result e.g. when trying to express recombinant proteins in fungal expression systems.

Cryptic introns can be identified by the use of Reverse Transcription Polymerase Chain Reaction (RT-PCR). In RT-PCR, mRNA is reverse transcribed into single stranded cDNA that can be PCR amplified to double stranded cDNA. PCR primers can then be designed to amplify parts of the single stranded or double stranded cDNA, and sequence analysis of the resulting PCR products compared to the sequence of the genomic DNA reveals the presence and exact location of cryptic introns (T. Kumazaki et al. (1999) J. Cell. Sci. 112, 1449-1453).

According to one embodiment of the invention the modification introduced into the wild type gene sequence will optimize the mRNA for expression in a particular host organism. In the present invention the host organism or host cell comprises fungi.

### Modified Nucleotide Sequences:

The modified nucleic acid sequences according to the invention originate from gram negative bacteria and in particular from Enterobacteria. In a particular embodiment the Enterobacterium is selected from the group consisting of Echerichia sp. and Citrobacter sp.

More particularly the gram negative bacterium is selected from Esherichia sp and Citrobacter sp.

Even more particularly the modified nucleic acid sequences according to the invention originate from E. coli.

In another particular embodiment modified nucleic acid sequences according to the invention originate from the group of Citrobacter sp consisting of Citrobacter braakii, Citrobacter amalonaticus, Citrobacter gillenii.

In one aspect of the present invention the modified nucleotide sequence encodes a hydrolase, more particularly the hydrolase is in one embodiment a phytase.

Particularly the wild type nucleic acid sequences to be modified according to the invention are the specific sequences shown in SEQ ID NO: 1, 3, and 4.

After modifying the wild type nucleic acid sequence, the polypeptide can be expressed in the host cell.

In a particular embodiment, the modified nucleic acid sequence encoding a wild type phytase polypeptide is selected from the group consisting of SEQ ID NO: 2, 6, 8, 61 and 62, particularly, the part encoding the mature phytase polypeptide. More particularly the modified nucleic acid sequence is selected from the group consisting of position 67 to 1302 in SEQ ID NO: 2, position 1 to 1236 in SEQ ID NO: 6, position 256 to 1491 in SEQ ID NO: 8, position 106 to 1341 in SEQ ID NO: 61, and position 106 to 1341 in SEQ ID NO: 62.

In the present context the term “capable of expression in a filamentous host” means that the yield of the phytase polypeptide should be at least 1.5 mg/l, more particularly at least 2.5 mg/l, more particularly at least 5 mg/l, more particularly at least 10 mg/l, even more particularly at least 20 mg/l, or more particularly 0.5 g/L, or more particularly 1 g/L, or more particularly 5 g/L, or more particularly 10 g/L, or more particularly 20 g/L.

Specific examples of modified nucleic acid sequences encoding phytases modified according to the invention in order to provide expression of the phytase polypeptide in a fungal host, like e.g. Aspergillus or Pichia, are shown in SEQ ID NO: 2, 6, 8, 61, and 62 The information disclosed herein will allow the skilled person to isolate other modified nucleic acid sequences following the directions above, which sequences can also be expressed in fungi and such sequences are also comprised within the scope of the present invention.

The choice of codon usage can be varied according to the desired host cell and the number of codons, which have been optimized can also vary and still provide a nucleic acid sequence capable of expression in a filamentous fungus. Such alternative sequences will be homologous to at least the part encoding the mature polypeptide in the specific sequences comprised in SEQ ID NO: 2, 6, 8, 61, and 62. Even starting from the same wild type nucleic acid sequence and employing the same codon table and the same modification protocol as described earlier the resulting modified nucleic acid sequences can vary due to the stochastic nature of the optimization process. Therefore among the resulting modified sequences it is usual to observe sequences variations up to about 20%. This means that the modified sequences based on the same wild type sequence will have a degree of identity of about 80% or more. In one embodiment the % identity is at least 83%, more particularly at least 85%, even more particularly at least 88%, and particularly at least 90%, even more particularly at least 95%, and most particularly at least 98%. SEQ ID NO: 2, 61 and 62 represents such variation with the difference that in SEQ ID NO: 2 the original signal peptide has been maintained (positions 1 to 66), whereas in SEQ ID NO: 61 and 62 the original signal peptide has been replaced with the Humicola insolens cutinase prepro signal (positions 1 to 105).

In a further embodiment the invention therefore relates to a modified nucleic acid sequence encoding the phytase polypeptide and capable of expression in a filamentous fungal host organism, wherein:

a) the modified sequence has at least 80% identity with position 67 to 1302 in SEQ ID NO: 2; or
b) the modified sequence hybridizes under medium stringency conditions with a polynucleotide probe consisting of the nucleotides 67 to 1302 of SEQ ID NO: 2; or the complementary strand thereof.

The modified nucleic acid sequence according to the invention therefore has at least 80% identity with the above sequence comprised in SEQ ID NO: 2, particularly at least 83%, more particularly at least 85%, more particularly at least 88%, even more particularly at least 90% identity, even more particularly at least 95%, and even most particularly at least 98%.

The present invention also relates to isolated polynucleotides encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 67 to 1302 of SEQ ID NO: 2, or (ii) a complementary strand of (i); or allelic variants and subsequences thereof (Sambrook et al., 1989, supra), as defined herein.

In another embodiment the invention therefore relates to a modified nucleic acid sequence encoding the phytase polypeptide and capable of expression in a filamentous fungal host organism, wherein:

a) the modified sequence has at least 80% identity with position 1 to 1236 in SEQ ID NO: 6; or
b) the modified sequence hybridizes under medium stringency conditions with a polynucleotide probe consisting of the nucleotides 1 to 1236 of SEQ ID NO: 6; or the complementary strand thereof.

The modified nucleic acid sequence according to the invention therefore has at least 80% identity with the above sequence comprised in SEQ ID NO: 6, particularly at least 83%, more particularly at least 85%, more particularly at least 88%, even more particularly at least 90% identity, even more particularly at least 95%, and even most particularly at least 98%.

The present invention also relates to isolated polynucleotides encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 1 to 1236 of SEQ ID NO: 6, or (ii) a complementary strand of (i); or allelic variants and subsequences thereof (Sambrook et al., 1989, supra), as defined herein.

In an even further embodiment the invention therefore relates to a modified nucleic acid sequence encoding the phytase polypeptide and capable of expression in a filamentous fungal host organism, wherein:

a) the modified sequence has at least 80% identity with position 256 to 1491 in SEQ ID NO: 8; or
b) the modified sequence hybridizes under medium stringency conditions with a polynucleotide probe consisting of the nucleotides 256 to 1491 of SEQ ID NO: 8; or the complementary strand thereof.

The modified nucleic acid sequence according to the invention therefore has at least 80% identity with the above sequence comprised in SEQ ID NO: 8, particularly at least 83%, more particularly at least 85%, more particularly at least 88%, even more particularly at least 90% identity, even more particularly at least 95%, and even most particularly at least 98%.

The present invention also relates to isolated polynucleotides encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 256 to 1491 of SEQ ID NO: 8, or (ii) a complementary strand of (i); or allelic variants and subsequences thereof (Sambrook et al., 1989, supra), as defined herein.

In another further embodiment the invention relates to a modified nucleic acid sequence encoding the phytase polypeptide and capable of expression in a filamentous fungal host organism, wherein:

a) the modified sequence has at least 80% identity with position 106 to 1341 in SEQ ID NO: 61; or
b) the modified sequence hybridizes under medium stringency conditions with a polynucleotide probe consisting of the nucleotides 106 to 1341 of SEQ ID NO: 61; or the complementary strand thereof.

The modified nucleic acid sequence according to the invention therefore has at least 80% identity with the above sequence comprised in SEQ ID NO: 61, particularly at least 83%, more particularly at least 85%, more particularly at least 88%, even more particularly at least 90% identity, even more particularly at least 95%, and even most particularly at least 98%.

The present invention also relates to isolated polynucleotides encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 106 to 1341 of SEQ ID NO: 61, or (ii) a complementary strand of (i); or allelic variants and subsequences thereof (Sambrook et al., 1989, supra), as defined herein.

In still another embodiment the invention relates to a modified nucleic acid sequence encoding the phytase polypeptide and capable of expression in a filamentous fungal host organism, wherein:

a) the modified sequence has at least 80% identity with position 106 to 1341 in SEQ ID NO: 62; or
b) the modified sequence hybridizes under medium stringency conditions with a polynucleotide probe consisting of the nucleotides 106 to 1341 of SEQ ID NO: 62; or the complementary strand thereof.

The modified nucleic acid sequence according to the invention therefore has at least 80% identity with the above sequence comprised in SEQ ID NO: 62, particularly at least 83%, more particularly at least 85%, more particularly at least 88%, even more particularly at least 90% identity, even more particularly at least 95%, and even most particularly at least 98%.

The present invention also relates to isolated polynucleotides encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 106 to 1341 of SEQ ID NO: 62, or (ii) a complementary strand of (i); or allelic variants and subsequences thereof (Sambrook et al., 1989, supra), as defined herein.

Particularly the modified nucleic acid sequences according to the invention consist of the sequences selected from the group consisting of SEQ ID NO: 2, 6, 8, 61, 62.

For purposes of the present invention, hybridization indicates that the nucleotide sequence hybridizes to a labelled nucleic acid probe corresponding to the nucleotide sequence detailed above, its complementary strand, or a subsequence thereof, under very low to very high stringency conditions. Molecules to which the nucleic acid probe hybridizes under these conditions can be detected using X-ray film.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

In a particular embodiment, the wash is conducted using 0.2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency). In another particular embodiment, the wash is conducted using 0.1×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

### Nucleic Acid Constructs

The present invention also relates to nucleic acid constructs comprising an isolated polynucleotide of the present invention operably linked to one or more control sequences which direct the expression of the coding sequence in a suitable host cell under conditions compatible with the control sequences.

The control sequence may be an appropriate promoter sequence, a nucleotide sequence which is recognized by a host cell for expression of a polynucleotide encoding a poly-peptide of the present invention. The promoter sequence contains transcriptional control sequences which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1,ADH2/GAP), Saccharomyces cerevisiae triose phosphate isomerase (TPI), Saccharomyces cerevisiae metallothionine (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans those phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, Humicola lanuginosa lipase, Humicola insolens cutinase (WO 2005121333), Candida albicans lipase B (CLB), Candida antarctica lipase B (CLB′), Fusarium solani lipase, Thermomyces lanuginosus lipase (WO 97/04079).

In a preferred aspect, the signal peptide coding region is nucleotides 1 to 54 of SEQ ID NO: 9 (CLB′), nucleotides 1-54 of SEDQ ID NO: 10 (CLB), nucleotides 1-54 of SEQ ID NO: 11 (H. isolens cutinase), or nucleotides 1-66 of SEQ ID NO: 56.

Useful signal peptides for yeast host cells are obtained from the genes for Saccharomyces cerevisiae alpha-factor and Saccharomyces cerevisiae invertase. Other useful signal peptide coding regions are described by Romanos et al., 1992, supra.

In a preferred aspect, the signal peptide coding region is the alpha-factor signal sequence shown in SEQ ID NO: 12 encoding the alpha signal peptide from S. cerevisiae.

The signal peptide encoding nucleic acid sequence may in one embodiment also be codon optimized according to the invention. The signal peptide may in one embodiment be codon optimized for expression in Pichia pastoris. This could e.g. result in the sequences shown in SEQ ID NO: 13 or nucleotides 1 to 255 in SEQ ID NO: 8.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Saccharomyces cerevisiae alpha-factor, Rhizomucor miehei aspartic proteinase, Myceliophthora thermophila laccase (WO 95/33836), Humicola insolens cutinase (WO 2005121333), Candida albicans lipase B (CLB) or Candida antarctica lipase B (CLB′)

In a preferred aspect, the propeptide coding region consists of nucleotides 55 to 75 of SEQ ID NO: 9 (CLB′), nucleotides 55 to 75 of SEQ ID NO: 10 (CLB), or nucleotides 55 to 105 of SEQ ID NO: 11 (H. insulens cutinase).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the polypeptide would be operably linked with the regulatory sequence.

### Expression Vectors

The present invention also relates to recombinant expression vectors comprising a polynucleotide of the present invention, a promoter, and transcriptional and translational stop signals. The various nucleic acids and control sequences described above may be joined together to produce a recombinant expression vector which may include one or more convenient restriction sites to allow for insertion or substitution of the nucleotide sequence encoding the polypeptide at such sites. Alternatively, a nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyl-transferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98:61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into the host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et at., 1989, supra).

### Host Cells

The present invention also relates to recombinant fungal host cells, comprising a polynucleotide of the present invention, which are advantageously used in the recombinant production of the polypeptides. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication.

“Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred aspect, the yeast host cell is a Candida, Hansenula, Kluyveromyces, Pichia, Saccharomyces, Schizosaccharomyces, or Yarrowia cell.

In a most preferred aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. In another most preferred aspect, the yeast host cell is a Kluyveromyces lactis cell. In another most preferred aspect, the yeast host cell is a Yarrowia lipolytica cell. In another most preferred aspect the host cell is a Pichia pastoris cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred aspect, the filamentous fungal host cell is an Acremonium, Aspergillus, Aureobasidium, Bjerkandera, Ceriporiopsis, Coprinus, Coriolus, Cryptococcus, Filobasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, or Ceriporiopsis subvermispora, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

The present invention relates to methods for producing a polypeptide of the present invention, comprising (a) cultivating a host cell under conditions conducive for production of the polypeptide; and (b) recovering the polypeptide.

In the production methods of the present invention, the cells are cultivated in a nutrient medium suitable for production of the polypeptide using methods well known in the art. For ex-ample, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered using methods known in the art. For ex-ample, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

### Materials and Methods

### Strains and Plasmids

The following expression hosts were used: A. oryzae strain BECh2 described in WO 00/39322, example 1, which is further referring to JaL228 described in WO 98/12300, example 1, genotype: amy⁻, alp⁻, Npl⁻, CPA⁻, KA⁻; A. niger strain MBin118 (described in WO2004090155 A2, example 11), genotype: AMG, ASA, NA1, NA2, prtT, tgs; Pichia pastoris strain KM71, genotype: arg4, his4, aox1::ARG4. E. coli DH5alpha (Invitrogen™), TOP10 (Invitrogen™) or XL10 (Stratagene) was used as cloning host in construction of the expression vectors. The expression plasmid pDAu104 (the same as pDAu109 (see reference below) except there is no signal sequence following the promoter and the polylinker is: BamHI, Acc651, Asp718, KpnI, AosI, AviII, FspI, SpeI, MluNI, MscI) containing the A. nidulans amdS gene as selection marker in Aspergillus and the Ampicillin resistance gene for selection in E. coli, two copies of the A. niger NA2 promoter (neutral amylase) with 3 extra amyR-sites+the 5′ untranslated part of the A. nidulans TPI promoter for heterologous expression and the A. niger AMG terminator, as well as pDAu109 (described in patent WO 2005/042735A1), which differs from pDAu104 in containing the Candida antarctica lipase B (CLB′) signal coding sequence, positions 1-54 of SEQ ID NO: 9, downstream of the NA2TPI promoter, was used. pPIC9K (Invitrogen) was used as cloning vector for expression in Pichia pastoris, it contains HIS4 as selection marker in Pichia pastoris and the AMP gene for selection in E. coli, expression is driven by the AOX1 promoter and terminator, the alpha factor secretion signal is downstream of the AOX1 promoter.

E. coli DH12S is available from Gibco BRL.

### PCR Amplification:

| | |
|---|---|
| 10 x PCR buffer (incl. MgCl₂) | 5 μl |
| 2.5 mM dNTP mix | 5 μl |
| Forward primer (10 μM) | 5 μl |
| Reverse primer (10 μM) | 5 μl |
| Expand High Fidelity polymerase (Roche) | 0.5 μl |
| Template DNA | 1 μl |
| Add autoclaved, distilled water to | 50 μl |

### Conditions

| | | | |
|---|---|---|---|
| 95° C. | 2 | min | 1 cycle |
| 94° C. | 30 | sec | |
| 55° C. | 30 | sec | 40 cycles (30 cycles for “synth. gene + clb |
| | | | signal” PCR) |
| 72° C. | 1.30 | min | |
| 72° C. | 2 | min | 1 cycle |

Transformation of Aspergillus: Transformation of BECh2 and MBin118 were performed by a method involving protoplast formation and transformation of these. Suitable procedures for Aspergillus transformation are described in EP 0 238 023 and Yelton et al., 1984, Proceedings of the National Academy of Sciences USA 81: 1470-1474. Transformants were isolated and grown in small Nunc-containers in 10 ml of YPM (1% yeast extract, 2% Bacto peptone, and 2% maltose) for 3 days at 30° C. (rotated).

Transformation of Pichia pastoris: Pichia pastoris were transformed by electroporation according to the manufacturers protocol (Invitrogen, Cat. #K1710-01). P. pastoris transformants were grown in BMGY medium for 3 days and spun down, followed by replacement of medium with BMMY (including methanol for induction of promoter). The cultures were allowed to grow for 2 more days, with addition of 0.3 ml methanol after one day.

SDS-page gel electrophoresis: 7.5 μl supernatant samples from the above described 10 ml cultures were subjected to SDS-gel electrophoresis. Gels were stained with Comassie.

Phytase activity—plate assay: 20 μL supernatant from 2-5 days incubation of the transformants was removed and applied into a 4 mm hole punched in the following plate: 1% agarose plate containing 0.1 M Sodium acetate (pH 4.5) and 0.1% Inositol Hexaphosphoric acid. The plate was incubated at 37° C. over night and a buffer consisting of 1M CaCl₂ and 0.2M Sodium acetate (pH 4.5) was poured over the plate. The plate was left at room temperature for 1 hr and the phytase activity identified as a clear zone.

### EXAMPLES

### Example 1

### Subcloning and Heterologous Expression of Citrobacter braakii Phytase in A. Oryzae—Wild Type Gene and Synthetic Variants

Construction of pPFJo202: The Citrobacter braakii phytase gene (SEQ ID NO: 1, entire ORF including predicted signal sequence) was amplified using the primers 304/Citrob-ND002281-wt-forw and 303/Citrob-ND002281-rev (designed from the full sequence) and genomic DNA from the Citrobacter braakii strain ATCC51113 (American Type Culture Collection) as template. This gives a 1334 base pair product. The primers have cloning restriction sites BamHI-XhoI, respectively, in the ends, as well as 15 by homology to the expression vector pDAu104, enabling cloning via the In-Fusion™ PCR cloning method, which is a restriction enzyme independent way of cloning (BD Biosciences, Cat # 631774). A pool of PCR product from individual PCR reactions was used for the cloning. The PCR product was purified from a gel using JetSorb (GENOMED) and cloned into pDAu104, digested with BamHI and XhoI, through the In-Fusion method. The insert was sequenced and verified to be identical to the original sequence.

Construction of pPFJo204: The Citrobacter braakii phytase gene without signal sequence (position 67-1302 in SEQ ID NO: 1) was amplified using the primers 302/Citrob-ND002281-(-sig)-forw and 303/Citrob-ND002281-rev (designed from the full sequence) and genomic DNA from the strain ATCC51113 as template. This gives a 1267 by fragment. The primers have cloning restriction sites FspI-XhoI, respectively, in the ends, as well as 15 by homology to the expression vector pDAu109, enabling cloning via the InFusion method (BD Biosciences). A pool of PCR product from individual PCR reactions was used for the cloning. The PCR product was purified from a gel using JetSorb (GENOMED) and cloned into pDAu109, digested with FspI and XhoI, through the InFusion method. The insert was sequenced and verified to be identical to the original sequence.

Construction of pPFJo217: The full length synthetic phytase gene (SEQ ID NO: 2 entire ORF including predicted signal sequence) was synthesized at DNA 2.0 (DNA 2.0 USA, 1430 O'Brian Drive, Suite E, Menlo Park, Calif. 94025 USA) and cut by SpeI-HindIII restriction enzymes from a plasmid, pJ1:G01249, purified from a gel using JetSorb as a 1310 base pair fragment and sub-cloned into pDAu104 digested with SpeI-HindIII. The synthetic gene was designed according to the codon table shown in Table 1 and according to the general rules described above. In addition to that the synthetic gene sequence was selected as one that did not give rise to prediction of introns when run through the intron prediction programme NetGene2 (Hebsgaard et al., Nucleic Acids Research, 1996, Vol. 24, No. 17, 3439-3452). Using the principles described herein any gene can be modified and synthesized accordingly. The skilled person will know how to clone synthetic genes designed according to the present invention into appropriate expression vectors.

Construction of pPFJo218: The synthetic phytase gene without signal (position 67-1302 in SEQ ID NO: 2) was amplified using the primers 323/s-cit.phyt-sig-forty and 324/s-cit.phyt-sig-rev (designed from the full sequence) from the template pJ1:G01249—this resulted in a 1265 base pair PCR product. The primers have cloning restriction sites AviII-HindIII, respectively, in the ends, as well as 15 by homology to the expression vector pDAu109, enabling cloning via the InFusion method (BD Biosciences). A pool of PCR product from individual PCR reactions was used for the cloning. The PCR product was purified from a gel using JetSorb and cloned into pDAu109, digested with AviII and HindIII using the In-Fusion method (BD Biosciences). The insert was sequenced and verified to be identical to the original sequence.

### Primers:

SEQ ID NO: 14

302/Citrob-ND002281-(-sig)-forw
CCTTTGGTGAAGTGCGAAGAGCAGAATGGTATGAAAC

SEQ ID NO: 15

303/Citrob-ND002281-rev
CCCTCTAGATCTCGAGTTATTCCGTAACTGCACACTC

SEQ ID NO: 16

304/Citrob-ND002281-wt-forw
ACACAACTGGGGATCCACCATGAGTACATTCATCATTCG

SEQ IN NO: 17

323/s-cit.phyt-sig-forw
CCTTTGGTGAAGTGCGAAGAGCAGAACGGAATGAAG

SEQ ID NO: 18

324/s-cit.phyt-sig-rev
AGATCTCGAGAAGCTTACTCTGTGACGGCAC

### Cloning and Transformation in Cloning and Transformation in Aspergillus

The expression plasmids pPFJo202, pPFJo204, pPFJo217 and pPFJo218 were made as de-scribed above. The plasmids were transformed into BECh2 (Aspergillus oryzae) and MBin118 (Aspergillus niger). Between 7 and 20 transformants were isolated, grown in YPM for 3 days and supernatants run on an SDS-PAGE. This showed varying expression levels ranging from nothing to quite good expression—see table 3 for an expression summary. The predicted molecular weight is 46 kDa, however, the actual molecular weight is 60-70 kDa and highly glycosylated. Supernatant from the best producing ones were applied onto phytase activity plates and all the tested transformants show phytase activity.

TABLE 3
An overview of the results of expressing Citrobacter braakii
phytase in Aspergillus

| | BECh2 | MBin118 |
|---|---|---|
| Expression plasmids transformed | (Aspergillus | (Aspergillus |
| into Aspergillus | oryzae) | niger) |
| pPFJo202 (wt gene + wt signal) | No expression | No expression |
| pPFJo204 (wt gene + clb′ signal) | No expression | No expression |
| pPFJo217 (synth. gene + wt signal) | Low expression | Very low |
| | | expression |
| pPFJo218 (synth. gene + clb′ signal) | Good expression | Low expression |

### Example 2

### Subcloning and Heterologous Expression of Citrobacter amalonaticus and Citrobacter gillenii Wild Type Phytase in A. Oryzae

### PCR Amplification/Conditions

As in described in Material and Methods—though the annealing temperature was 60° C. and the cycle number was 25 for the full length Citrobacter amalonaticus phytase product and 35 for the same phytase without signal. For both Citrobacter gillenii phytase PCR products were used a cycle number of 40.

Construction of pPFJo177: The Citrobacter amalonaticus phytase gene (SEQ ID NO: 3 entire ORF including predicted signal sequence) was amplified by PCR using the primers 258/Citrobacter phyt rev2 and 259/Citrobacter phyt forw3 (designed from the full sequence) and genomic DNA from the strain Citrobacter amalonaticus ATCC25405 (American Type Culture Collection) as template. This results in a 1346 base pair product. The primers have cloning restriction sites BamHI-XhoI, respectively, in the ends, as well as 15 by homology to the expression vector pDAu104, enabling cloning via the InFusion method (BD Biosciences). A pool of PCR product from individual PCR reactions was used for the cloning. The PCR product was purified from a gel using JetSorb (GENOMED) and cloned into pDAu104, digested with BamHI and XhoI, through the InFusion method. The insert was sequenced and verified to be identical to the original sequence.

Construction of pPFJo178: The Citrobacter amalonaticus phytase gene without signal sequence (Sequence ID 3 position 67-1311) was amplified by PCR using the primers 257ny/Citrobacter phyt forw1 and 258/Citrobacter phyt rev2 and genomic DNA from the strain Citrobacter amalonaticus ATCC25405 (American Type Culture Collection) as template. This results in a 1276 base pair product. The primers have cloning restriction sites FspI-XhoI, respectively, in the ends, as well as 15 by homology to the expression vector pDAu109, enabling cloning via the InFusion method (BD Biosciences). A pool of PCR product from individual PCR reactions was used for the cloning. The PCR product was purified from a gel using JetSorb (GENOMED) and cloned into pDAu109, digested with FspI and XhoI, through the InFusion method. The insert was sequenced and verified to be identical to the original sequence.

Construction of pPFJo203: The Citrobacter gillenii phytase gene (SEQ ID NO: 4, entire ORF including predicted signal sequence) was amplified using the primers 307/Citrobac-ND002284-wt-forw and 306/Citrobac-ND002284-rev and genomic DNA from the strain DSM 13694 (DSMZ-Deutche Sammlung von Mikroorganismen and Zellkulturen GmbH) as template. This results in a 1332 base pair product. The primers have cloning restriction sites BamHI-XhoI, respectively, in the ends, as well as 15 by homology to the expression vector pDAu104, enabling cloning via the In-Fusion method (BD Biosciences). A pool of PCR product from individual PCR reactions was used for the cloning. The PCR product was purified from a gel using JetSorb (GENOMED) and cloned into pDAu104, digested with BamHI and XhoI, through the InFusion method. The insert was sequenced and verified to be identical to the original sequence.

Construction of pPFJo205: The Citrobacter gillenii phytase gene without signal sequence (SEQ ID NO: 4, position 67-1299) was amplified using the primers 305/Citrob-ND002284-(-sig)-forw and 306/Citrobac-ND002284-rev and genomic DNA from the strain NN019345 as template. This results in a 1264 base pair product. The primers have cloning restriction sites FspI-XhoI, respectively, in the ends, as well as 15 by homology to the expression vector pDAu109, enabling cloning via the InFusion method (BD Biosciences). A pool of PCR product from individual PCR reactions was used for the cloning. The PCR product was purified from a gel using Jet-Sorb (GENOMED) and cloned into pDAu109, digested with FspI and XhoI, through the InFusion method. The insert was sequenced and verified to be identical to the original sequence.

### Primers:

SEQ ID NO: 19

257ny/Citrobacter phyt forw1
CCTTTGGTGAAGTGCGAAGTGCCAGATGACATGAAGC

SEQ ID NO: 20

258/Citrobacter phyt rev2
CCCTCTAGATCTCGAGTTAACGGTTTACATCAGCCATC

SEQ ID NO: 21

259/Citrobacter phyt forw3
ACACAACTGGGGATCCACCATGAATACGCTACTTTTTCG

SEQ ID NO: 22

305/Citrob-ND002284-(-sig)-forw
CCTTTGGTGAAGTGCGATGAACAGAGCGGAATGCAGC

SEQ ID NO: 23

306/Citrobac-ND002284-rev
CCCTCTAGATCTCGAGTTATTTCTCAGCACATTCGGACAC

SEQ ID NO: 24

307/Citrobac-ND002284-wt-forw
ACACAACTGGGGATCCACCATGAGTACACTGATCATTCG

Cloning and transformation in Aspergillus

The different Citrobacter phytase PCR products were cloned into pDAu104 and pDAu109 as described above, resulting in pPFJo177, pPFJo178, pPFJo203 and pPFJo205. All constructs were transformed into A. oryzae BECh2 and A. niger MBin118. Between 7 and 12 transformants with each construct in each host were isolated and inoculated in 10 ml YPM. Supernatant samples were run on SDS gels. From pPFJo177 and pPFJo178 in MBin118, bands are visible around the expected size of the phytase (˜46 kDa). Expression results are summarized in the table below.

TABLE 4
An overview of the results of expressing Citrobacter braakii
phytase in Aspergillus

| Expression plasmids | | | BECh2 | MBin118 |
|---|---|---|---|---|
| transformed into | Signal | Mature | (Aspergillus | (Aspergillus |
| Aspergillus | seq. | seq. | oryzae) | niger) |
| pPFJo177 | wt | wt | No expression | Low expression |
| (C. amalonaticus) | | | | |
| pPFJo178 | CLB′ | wt | No expression | Low expression |
| (C. amalonaticus) | | | | |
| pPFJo203 | wt | wt | No expression | No expression |
| (C. gillenii) | | | | |
| pPFJo205 | CLB′ | wt | No expression | No expression |
| (C. gillenii) | | | | |

### Example 3

### Expression of Synthetic Citrobacter braaki Phytase Gene in Aspergillus

### Aspergillus Transformation and Cultivation

The method for transformation of Aspergillus strains and selections and cultivation of Aspergillus transformants are described in WO 02/20730.

Aspergillus oryzae strain BECh2 was inoculated in 100 ml of YPG medium and incubated at 32° C. for 16 hours with stirring at 80 rpm. Grown mycelia was collected by filtration followed by washing with 0.6 M KCl and re-suspended in 30 ml of 0.6 M KCl containing Glucanex® (Novozymes) at the concentration of 30 μl/ml. The mixture was incubated at 32° C. with the agitation at 60 rpm until protoplasts were formed. After filtration to remove the remained mycelia, protoplasts were collected by centrifugation and washed with STC buffer twice. The protoplasts were counted with a hematitometer and re-suspended in a solution of STC:STPC:DMSO (8:2:0.1) to a final concentration of 1.2×10⁷ protoplasts/ml. About 4 μg of DNA was added to 100 μl of protoplast solution, mixed gently and incubated on ice for 30 minutes. 1 μl STPC buffer was added to the mixture and incubated at 37° C. for another 30 minutes. After the addition of 10 ml of Cove top agarose pre-warmed at 50° C., the reaction mixture was poured onto COVE-ar agar plates. The plates were incubated at 32° C. for 5 days.

### PCR Reaction

Unless otherwise indicated the PCR reactions were carried out under the following conditions: The PCR reaction contained 38.9 MicroL H2O, 5 MicroL 10× reaction buffer, 1 MicroL Klen Taq LA (Clontech), 4 MicroL 10 mM dNTPs, 0.3 MicroL×2 100 pmol/MicroL primer and 0.5 MicroL template DNA and was carried out under the following conditions: 30 cycles of 10 sec at 98° C. and 90 sec at 68° C., and a final 10 min at 68° C.

### Other Methods

DNA Plasmids were prepared with the Qiagen® Plasmid Kit. DNA fragments and recovered from agarose gel by the Qiagen gel extraction Kit.

PCR was carried out by the PTC-200 DNA Engine.

The ABI PRISMTM 310 Genetic Analyzer was used for determination of all DNA sequences.

### Phytase Assay

Ten microL diluted enzyme samples (diluted in 0.1 M sodium acetate, 0.01% Tween20, pH 5.5) were added into 250 microL of 5 mM sodium phytate (Sigma) in 0.1 M sodium acetate, 0.01% Tween20, pH 5.5 (pH adjusted after dissolving the sodium phytate; the substrate was preheated) and incubated for 30 minutes at 37° C. The reaction was stopped by adding 250 microL 10% TCA and free phosphate was measured by adding 500 microL 7.3 g FeSO4 in 100 ml molybdate reagent (2.5 g (NH4)₆Mo7024.4H20 in 8 ml H2SO4 diluted to 250 ml). The absorbance at 750 nm was measured on 200 MicroL samples in 96 well microtiter plates. Substrate and enzyme blanks were included. A phosphate standard curve was also included (0-2 mM phosphate). 1 U equals the amount of enzyme that releases 1 micromol phosphate/min at the given conditions.

### Media

MS-9: per liter 30 g soybean powder, 20 g glycerol, pH 6.0.

MDU-2 Bp: per liter 45 g maltose-1H2O, 7 g yeast extract, 12 g KH2PO4, 1 g MgSO4-7H2O, 2 g K2SO4, 5 g Urea, 1 g NaCl, 0.5 ml AMG trace metal solution pH 5.0.

### Primers

CutisignalF,

SEQ ID NO: 25

CAACTGGGGATCTGGTACCACCATGAAGTTCTTCACCACC
Cutipre-EER,

SEQ ID NO: 26

CTTCATTCCGTTCTGCTCTTCGGGGAGAGCAGCAACAAGGC
Cutiprepro-EER,

SEQ ID NO: 27

CTTCATTCCGTTCTGCTCTTCCCGGGCAACAAGTTCAGGAG
EEF,

SEQ ID NO: 28

GAAGAGCAGAACGGAATGAAG
CitroC-termR,

SEQ ID NO: 29

CAGTCACCCTCTAGATCTCGACTTAATTAACTACTCTGTGACGGCACAC

Humicola insolens Cutinase signal peptide encoding sequence (SEQ ID NO: 11, nucleotides 1-54) or the signal sequence and pro sequence (SEQ ID NO: 11) were amplified with primer pairs, cutisignalF and cutipreEER or cutisignalF and cutipreproEER, using pTM-TPcutiprepro, which is described in WO2005121333, as template. Mature region of synthetic Citrobacter braaki phytase gene was amplified with a primer pair, EEF and citroC-term R, using pPFJo217 as template. Both the obtained PCR fragments were recovered from agarose gel, cut with KpnI and XhoI, and introduced into pAEY039 amp digested with KpnI and XhoI.

The plasmid pAEY039 amp pAEY039 amp is a derivative of plasmid pMT2188 described in WO 03/089648 (Example 24 page 47). It has a KpnI site instead of a BamHI site. Also, it has an ampicilin gene and an E. coli replication origin, position 454 bp to 2686 bp in pUC19 (TAKARA), instead of a 1353 bp of SbfI fragment which contains E. coli replication origin in pMT2188. Plasmid pMT2188 comprises an expression cassette based on the Aspergillus niger neutral amylase II promoter fused to the Aspergillus nidulans triose phosphate isomerase non translated leader sequence (Na2/tpi promoter) and the Aspergillus niger amyloglycosidase terminator (AMG terminator), the selective marker amdS from Aspergillus nidulans enabligng the growth on acetamide as sole nitrogen source, and the URA3 marker from Saccharomyces cerevisiae enabling growth on the pyrF defective Escherichia coli strain DB6507.

The Humicola signal and pro regions were amplified by PCR using pTM-TPcutiprepro as template as described above. Then, using the PCR fragment of signal or singal+pro and the PCR fragment of amplified Citrobactor phytase mature sequence, SOE-PCR (splicing by overlap extension PCR) was carried out with a primer pair of cutisignalF and citroC-termR. The obtained PCR fragment containing signal+(pro)+Citrobactor phytase was recovered from agarose gel and digested with KpnI and XhoI. Plasmid preparation was carried out in E. coli DH12S. Resulting plasmids were termed pCBPhycuti and pCBPhycutiprepro.

pCBPhycuti and pCBPhycutiprepro were introduced into Aspergillus oryzae Becht and the obtained transformants were cultivated in MS-9 medium followed by MDU-2 Bp medium. The phytase activities of the supernatants of each transformants were determined.

TABLE 5
Expression results

| Strain | signal | pro | Mature seq. | Expression |
|---|---|---|---|---|
| PFJo205 | CLB′ | | synthetic | Good |
| | | | | expression |
| pCBPhycutipre | Humicola | | synthetic | Good |
| | cutinase | | | expression |
| | signal | | | |
| pCBPhycutiprepro | Humicola | Humicola | synthetic | Very good |
| | cutinase | cutinase | | expression |
| | signal | | | |

### Example 4

### Expression of Wild Type Citrobacter braakii Phytase in Pichia pastoris

### Host Strain:

Pichia pastoris KM71 (from Invitrogen™)

### GS115 (Invitrogen™, Multi-Copy Pichia Expression Kit, Cat#:25-0170)

### Vectors:

pPIC9K; Pichia pastoris expression vector with alpha-factor secretion signal, SEQ ID NO: 12, under AOX1 promoter.
pPFJo202; Cit phyt ND002281—wt, Citrobacter braakii phytase construct from Example 1.

### PCR Primers

| Oligo | |
|---|---|
| Name | Oligo Seq |
| A-Na | GATCCAAACCATGagatttccttcaattttCac |
| | (SEQ ID NO: 30) |
| A-Nb | CAAACCATGagatttccttcaattttCac |
| | (SEQ ID NO: 31) |
| APhy-R | TCTGCTCTTCTCTTTTCTCGAGAGATACCCCTTC |
| | (SEQ ID NO: 32) |
| APhy-F | ctcgagaaaagaGAAGAGCAGAATGGTATGAAACTTG |
| | (SEQ ID NO: 33) |
| Phy-Ca | AATTCTTATTCCGTAACTGCACACTCTGG |
| | (SEQ ID NO: 34) |
| Phy-Cb | CTTATTCCGTAACTGCACACTCTGG |
| | (SEQ ID NO: 35) |

### Media:

MD (1.34% YNB, 4×10⁻⁵% biotin, 2% dextrose)
BMSY (1% yeast extract, 2% peptone, 100 mM potassium phosphate buffer, pH 6.0, 1.34% YNB, 4×10⁻⁵% biotin, 1% sorbitol)
BMGY (1% yeast extract, 2% peptone, 100 mM potassium phosphate buffer, pH 6.0, 1.34% YNB, 4×10⁻⁵% biotin, 1% glycerol)
BMMY (1% yeast extract, 2% peptone, 100 mM potassium phosphate buffer, pH 6.0, 1.34% YNB, 4×10⁻⁵% biotin, 0.5% methanol)

### Phytase Activity Plate Assay:

20 μl of culture broth was applied into a 4 mm hole punched in the 1% agarose plate containing 0.2% phytic acid dodecasodium salt in 0.1M sodium acetate (pH5.5), The plate was incubated at 37° C. overnight. 0.1 M CaCl₂ in 0.2M sodium acetate (pH5.5) was overlaid on the plate for 30-60 min. The phytase activity was identified as a clear zone.

Phytase standard: Bio-feed phytase, batch 84-11401, 5191 FYT(V)/g:

88.2 mg of Bio-feed phytase was dissolved into 104 ml of 0.1M sodium acetate (pH5.5) to pre-pare stock solution of the standard (4.4 FYT(V)/ml).

### Construction of Expression Vectors:

The Pichia pastoris expression construct for Citrobacter braakii phytase, namely pPIC9K-WT cb phytase, was generated as follows: the PCR fragment encoding the mature form of cb phytase (SEQ ID NO: 5 corresponding to SEQ ID NO: 1 without the signal position 1-66) fused in-frame with α-factor signal peptide SEQ ID NO: 12, was created by overlap extension PCR method: the fragment 1 encoding the alpha-factor signal peptide was amplified from pPIC9K plasmid with specific primers A-Na and APhy-R, while the fragment 2 encoding mature phytase was amplified from plasmid pPFJo202- Cit phyt (SEQ ID NO: 5)—wt using specific primers APhy-F and Phy-Cb. Then fragment 1 and 2 were mixed and used as a template for second step PCR amplification with specific primers A-Na/b and Phy-Ca/b to obtain the targeted PCR fragment. The DNA fragment was purified by gel extraction kit and then subcloned into pPIC9K vector in the BamHI and EcoRI sites. The resulting expression construct was confirmed by sequencing.

### Yeast Transformation:

Pichia pastoris KM71 or GS115 was transformed using electroporation protocol, according to the Invitrogen manual. Competent cells were prepared as described and stored in 40 μl aliquots at −70° C. 5 μg of plasmid DNA was linearized with PmeI leading to insertion of the plasmid at the chromosomal 5′AOX1 locus. Linearized plasmid DNA (500 ng) was mixed with 40 μl of competent cells and stored on ice for 5 min. Cells were transferred to an ice-cold 0.2 cm electroporation cuvette. Transformation was performed using a BioRad GenePulser II. Parameters used were 1500 V, 25 λF and 200Ω. Immediately after pulsing, cells were suspended in 1 ml of ice cold 1 M sorbitol. The mixtures were plated on MD plates. Plates were incubated at 28° C. for 3-4 days. The transformation of pPIC9K-WT cb phytase (alpha-phytase) into Pichia pastoris KM71 and GS115 resulted in hundreds of transformants. A total of 48 selected transformants were re-streaked on MD plates and grown for 2 days before expression screening.

Screening Clones for Expression Pichia pastoris KM71 in a 3 ml Scale:

The 48 selected transformants of pPIC9K-WT cb phytase in Pichia pastoris KM71 were tested for the expression of the desired phytase protein. Expression test was done in a 3 ml scale using 24-deep well plates (Whatman, UK). Each transformant was grown in BMSY media for 2.5 days at 28° C. with vigorous shaking (200 rpm); then 300 μl 0.5% methanol was added to each well every day for 4 days to induce heterogeneous gene expression. Samples of medium culture were taken daily during induction, stored at −20° C. for SDS-PAGE analysis and phytase activity assay.

The culture supernatant was analyzed by using phytase plate assay as described above. The bioactive samples were run on SDS-PAGE gel for estimation of protein expression level. Among 48 tested transformants, clear band at expected size was observed in culture medium from 47 of transformants. Strong phytase activity was detected in culture broth of these transformants harvested after methanol induction. Four highly-expressed transformants (alpha-phytase #3, #8, #23 and #46), which also showed relatively high phytase activity were identified.

### Scale-Up Expression of the Selected Transformants in KM71 Strain:

500 ml of BMGY media were inoculated with each of the selected strains of interest (#2, 8, 23, 46) in a 2 liter shake flask and incubated with shaking at 220 rpm for 3 days at 28° C. Cells were pelleted and resuspended in 500 ml of BMMY at 28° C. with shaking; cells were grown for 3 days with a daily supplement of 0.5% methanol to maintain the secretion. After induction, culture from 4 flasks was harvested. Cells were removed by centrifugation. The proteins from supernatant were precipitated by addition of ammonium sulfate at 90% saturation on ice under slow stirring. Insoluble proteins were pelleted by centrifugation and stored at −20° C. for purification.

10 μl of the culture supernatant was analyzed by using phytase plate assay as described above and it was found that the expressed protein was in active form. The samples were also run on SDS-PAGE gel for estimation of protein expression level. Compared to the samples of mini-scale, recombinant proteins were expressed at a comparable level.

TABLE 6

| Strain | Signal Seq | Mature seq. | Expression in KM71 |
|---|---|---|---|
| α-signal-Cb- | wt | wt | Good expressed in both |
| wt-phytase | | | mini and large scale expression |

Expression of Wild Type Phytase in P. pastoris Strain GS115:

The expression construct pPIC9K-WT cb phytase was transformed into another P. pastoris strain GS115. Mut⁺ and Mut^{s} transformants were reisolated and tested by inoculating in 3 ml culture in 24 deep-well plates. The culture supernatant after 4 day induction with methanol was analyzed by phytase activity assay. The bioactive samples were run on SDS-PAGE gel for estimation of protein expression level. All 24 Mut⁺ transformants from wild type gene showed phytase activity, while only 33%-58% of tested Mut^{s} transformants displayed phytase activity. Compared to Mut⁺ transformants from wild type gene, Mut^{s} of wild type gene showed stronger phytase activity.

TABLE 7
Overview of wt cb phytase expression in
different pichia pastoris host

| Pichia pastoris | | | | |
|---|---|---|---|---|
| Host | Phenotype | Signal | Mature seq. | Expression |
| KM71 | Muts | wt | wt | good |
| GS115 | Muts | wt | wt | good |
| GS115 | Mut+ | wt | wt | low |

### Example 5

### Expression of Synthetic Citrobacter Braakii Phytase Gene in Pichia pastoris

### Vectors:

pPIC-NoT, Pichia pastoris expression vector under AOX1 promoter, which was derived by eliminating the alpha-secretion signal from pPIC9K.

To create pPIC-NoT vector plasmid pPIC9K was digested with BamHI and EcoRI, and the digested major fragment was isolated from agarose gel. A synthetic DNA fragment containing BamHI and EcoRI sites were created by annealing the following two oligoes:

| | | |
|---|---|---|
| NoT-1 | P-GATCCTACGTAGCTGAG | (SEQ ID NO: 36) |
| and | | |
| NoT-2 | P-AATTCTCAGCTACGTAG | (SEQ ID NO: 37) |

The above synthetic DNA fragment was ligated into the digested pPIC9K plasmid, and the resulting vector pPIC-NoT was verified by sequencing.

pJ2:G01651, containing the synthetic phytase construct generated by company DNA2.0 encoding mature form of C. braakii phytase.

### PCR Primers

| Oligo | |
|---|---|
| Name | Oligo Seq |
| OA-Na | GATCCAAACCATGAGATTCCCATCCATCTTCACTG |
| | (SEQ ID NO: 38) |
| OA-Nb | CAAACCATGAGATTCCCATCCATCTTCACTG |
| | (SEQ ID NO: 39) |
| OAPhy-R | CATTCTGTTCCTCTCTCTTTTCCAAGGAAACACCTTC |
| | (SEQ ID NO: 40) |
| OAPhy-F | ggaaaagagaGAGGAACAGAATGGAATGAAGTTGG |
| | (SEQ ID NO: 41) |
| OPhy-Ca | AATTCTTACTCGGTGACAGCGCACTC |
| | (SEQ ID NO: 42) |
| OPhy-Cb | CTTACTCGGTGACAGCGCACTC |
| | (SEQ ID NO: 43) |

### Host Strains:

Pichia pastoris KM71 (Mut^{s} His⁻)
Pichia pastoris GS115 (Mut⁺His⁻)

### Media:

MD (1.34% YNB, 4×10⁻⁵% biotin, 2% dextrose)
BMSY (1% yeast extract, 2% peptone, 100 mM potassium phosphate buffer, pH 6.0, 1.34% YNB, 4×10⁻⁵% biotin, 1% sorbitol)
BMGY (1% yeast extract, 2% peptone, 100 mM potassium phosphate buffer, pH 6.0, 1.34% YNB, 4×10⁻⁵% biotin, 1% glycerol)
BMMY (1% yeast extract, 2% peptone, 100 mM potassium phosphate buffer, pH 6.0, 1.34% YNB, 4×10⁻⁵% biotin, 0.5% methanol)

### Design of Synthetic Phytase Gene

In order to increase the expression yield of the cb phytase in Pichia pastoris, the wild type Citerbacter phytase gene was modified based on P. pastoris-preferred codon usage, by means of replacing rare codons, eliminating repetitive AT and decreasing the GC content. The de-signed sequence was also analyzed to avoid potential intron. The procedure was as describe herein.

The modified phytase genes (G01651) fused to a modified alpha-factor secretion signal sequence were designed based on the codon bias of P. pastoris. The P. pastoris codon usage table is from www.kazusajp as well as Zhao et al, 2000 (Zhao X, Huo K K, Li Y Y. Synonymous condon usage in Pichia pastoris. Chinese Journal of Biotechnology, 2000, 16(3): 308-311). Rare codons for arginine were eliminated. Besides substitution of rare codons, the total G+C content was decreased below 50%, and AT-rich regions were modified to avoid premature termination. In addition, cryptic introns within modified coding region were eliminated as described. The synthetic gene sequence is shown in SEQ ID NO: 6 (complete ORF without signal sequence).

### Yeast Transformation:

P. pastoris (KM71 or GS115) was transformed using electroporation protocol, according to the Invitrogen manual. Competent cells were prepared as described and stored in 40 μl aliquots at −70° C. 5 μg of plasmid DNA was linearized with proper restriction enzymes leading to insertion of the plasmid at the chromosomal 5′AOX1 locus. Linearized plasmid DNA (500 ng) was mixed with 40 μl of competent cells and stored on ice for 5 min. Cells were transferred to an ice-cold 0.2 cm electroporation cuvette. Transformation was performed using a BioRad GenePulser II. Parameters used were 1500 V, 25 μF and 200Ω. Immediately after pulsing, cells were suspended in 0.5 ml of ice cold 1 M sorbitol. The mixtures were plated on MD plates and then incubated at 28° C. for 3-4 days. The transformations Selected His⁺ transformants were restreaked on MD plates and grown for 2 days before expression screening.

### Screening Clones for Expression in a 3 ml Scale:

Expression test of the selected transformants was done in a 3 ml scale using 24-deep well plates (Whatman, UK). Each transformant was grown in BMSY media for 2.5 days at 28° C. with vigorous shaking (200 rpm); then 300 μl 0.5% methanol was added to each well every day for 4 days to induce heterogeneous gene expression. Samples of medium culture were taken daily during induction, stored at −20° C. for SDS-PAGE analysis and phytase activity assay.

### Phytase Activity Plate Assay:

10-20 μl of culture broth was applied into a 4 mm hole punched in the 1% agarose plate containing 0.2% phytic acid dodecasodium salt in 0.1M sodium acetate (pH5.5), The plate was incubated at 37° C. overnight. 0.1 M CaCl₂ in 0.2M sodium acetate (pH5.5) was overlaid on the plate for 30-60 min. The phytase activity was identified as a clear zone.

Phytase standard: Bio-feed phytase, batch 84-11401, 5191 FYT(V)/g:

88.2 mg of Bio-feed phytase was dissolved into 104 ml of 0.1M sodium acetate (pH5.5) to pre-pare stock solution of the standard (4.4 FYT(V)/ml).

### Construction of Expression Vectors Using RIC (Restriction Independent Cloning) Cloning Strategy

The expression vector pPICNoT-G01651 was generated according to the following procedure: the PCR fragment encoding the mature form, SEQ ID NO: 6, of cb phytase fused in-frame with optimized α-factor signal peptide, encoded by SEQ ID NO: 13, was created by overlap extension PCR method as follows: the fragment I containing α-factor signal peptide was amplified from pJ2:G01468 plasmid (pJ2:G01468 was generated by DNA2.0, and contains the mature form of plectasin fused with α-factor secretion signal which was modified based on P. pastoris codon usage) with specific primers OA-Na and OAPhy-R, while the fragment II encoding mature phytase was amplified from plasmid pJ2:G01651 using specific primers OAPhy-F and OPhy-Ca. Then fragment I and II were mixed and used as a template for 2^{nd} step PCR amplification with specific primers OA-Na/b and OPhy-Ca/b to obtain the targeted PCR fragment. The DNA fragment was purified by gel extraction kit then subcloned into pPICNoT vector at BamHI and EcoRI sites. The resulting expression construct was confirmed by sequencing.

Transformation and Expression Test in Pichia pastoris KM71:

The expression construct pPICNoT-G01651 (A-G01651) was transformed into Pichia pastoris KM71 according the method described above and this resulted in hundreds of transformants. 60 of the randomly selected transformants were reisolated and tested by inoculating in 3 ml culture in 24 deep-well plates. The transformants were grown for 2.5 days and induced for 4 days. The culture supernatant was analyzed by phytase activity assay. The bioactive samples were run on SDS-PAGE gel for estimation of protein expression level. Clear band at expected size was observed in all transformants except #48. Compared to strains from wild type gene, the expression level of phytase from synthetic gene is mush higher. Strong phytase activity was detected in culture broth of the 59 transformants from synthetic gene harvested after methanol induction. The phytase activity of best expressers from synthetic gene is about 2 fold increased.

TABLE 8
Expression data

| Pichia pastoris | | | | |
|---|---|---|---|---|
| Host | Phenotype | Signal | Mature seq. | Expression |
| KM71 | Muts | synthetic | synthetic | good |

Transformation and Expression Test in Pichia pastoris GS115:

The expression construct pPICNoT-G01651 (synthetic cb phytase) was transformed into Pichia pastoris strain GS115. Mut⁺and Mut^{s} transformants were reisolated and tested by inoculating in 3 ml culture in 24 deep-well plates. The culture supernatant after 4 day induction with methanol was analyzed by phytase activity plate assay. The bioactive samples, identified by the plate assay, were run on SDS-PAGE gel for estimation of protein expression level. All 24 Mut⁺transformants from wild type gene and from synthetic gene showed phytase activity, while only 33%-58% of tested Mut^{s} transformants displayed phytase activity. Compared to Mutt transformants of the synthetic gene, Mut^{s} of the synthetic gene showed stronger phytase activity. For both wild type and synthetic genes, the expression level of phytase in GS115 is lower than in KM71. The synthetic gene did further improve the protein yield in GS115, about 2 fold increase compared to the wild type gene (Table 9).

TABLE 9
Overview of synthetic cb phytase expression
in different Pichia pastoris host

| Pichia pastoris | | | | |
|---|---|---|---|---|
| Host | Phenotype | Signal | Mature seq. | Expression |
| KM71 | Muts | synthetic | synthetic | good |
| GS115 | Muts | synthetic | synthetic | good |
| GS115 | Mut+ | synthetic | synthetic | low |

### Example 6

### Lab-Scale Expression of Synthetic Citrobacter braakii Phytase Gene in Pichia pastoris KM71

### Strains:

Pichia pastoris KM71 harboring pPIC9K-wt cb phytase as described in Example 4.
Pichia pastoris KM71 harboring pPICNoT-G01651 (A-G01651) as described in Example 5.

### Media:

YPD medium: 10.0 g/l yeast extract, 20.0 g/l peptone and 20.0 g/l glucose.

Fermentation basal salts medium: 26.7 ml/l 85% H₃PO₄, 1.1 g/l CaSO₄.2H₂O, 18.2 g/l K₂SO₄, 14.9 g/l MgSO₄.7H₂O, 4.1 g/l KOH, 40 g/l glycerol and 4.35 ml/l PTM1 trace salts. PTM1 trace salts medium: 65.00 g/l FeSO₄.7H₂O, 6.00 g/l CuSO₄.5H₂O, 20.00 g/l ZnCl₂, 4.30 g/l MnSO₄.5H₂O, 0.92 g/l CoCl₂.6H₂O, 0.20 g/l Na₂MoO₄.2H₂O, 0.02 g/l H₃BO₃, 0.09 g/l KI, 0.20 g/l Biotin and 5.00 ml/l H₂SO₄.

### Fermentation Conditions:

Seed: 5-10 micro liter cryopreserved cells were inoculated into a 500 ml shake flask containing 110 ml of YPD. Seed cultivation was conducted at 30° C. for 24 hours on the rotary shaker at 220 rpm.

Fermentation conditions in tank: Throughout fermentation the temperature was kept at 30° C., pH was adjusted at 5.0 with 25% ammonium hydroxide. Air flow was constant at 5.0 l/min. Pressure was kept at 0.05 MPa. Dissolved oxygen concentration was prevented from falling below oxygen limitation by the agitation control.

Glycerol batch phase: 90 g seed culture was inoculated into a 5-liter tank containing 2 liter fermentation basal salts medium after pH was adjusted to 5.0 with 25% ammonium hydroxide.

Glycerol fed-batch phase: 65% glycerol including 8 ml/l PTM1 trace salts medium was dosed 12 hours from fermentation start. The glycerol dosing was initiated at 10 g/hr and ramped up to 51 g/hr in 24 hours. The glycerol fed-batch phase was terminated at 40 hours and changed to a methanol fed-batch phase.

Methanol fed-batch phase: 100% methanol including 12 ml/l PTM1 trace salts medium was fed from the 40 hours point. Methanol dosing was conducted preventing methanol toxicity and oxygen limitation.

### Determination of Phytase Activity

7.5 mM of sodium phytate dissolved in the acetate buffer, pH 5.5, is mixed with ½ volume of enzyme sample solution in the same acetate buffer containing 0.01% Tween 20. After incubation at 37° C. for 30 minutes, the stop reagent containing 20 mM ammonium heptamolybdate and 0.06% ammonium vanadate dissolved in 10.8% nitric acid is added to generate a yellow complex with released inorganic phosphate. The amount of released phosphate is measured photometrically as the absorbance at 405 nm. One phytase unit is defined as the amount of enzyme to release 1 μmol inorganic phosphate per minute.

Test of Codon Optimized Synthetic Gene Expression in Pichia pastoris KM71 in 5-Liter Scale Tank

The Citrobacter braakii phytase gene was modified for optimal expression in Pichia pastoris as described in example 5. The synthetic gene containing mature form of phytase fused to the α-factor signal peptide was sub-cloned and expressed in P. pastoris KM71. Of the obtained recombinants the best clone in the 24-well plate testing was tested in 5-liter tank together with the best recombinant clone harboring the wild type Citrobacter braakii phytase gene in KM71 as described in example 4.

Fermentations were carried out for 7 days. Supernatant sample was collected on day 3, 4, 5, 6 and 7 from each tank and phytase activity determined. Compared to the clone expressing the wild type gene, expression of the synthetic phytase gene gave 2 fold increase in phytase activity in all 5 days samples. The gene optimization was therefore an effective technology for yield improvement.

TABLE 10
Overview of wt and synthetic cb phytase
expression in 5-liter lab-scale tank

| Pichia pastoris | | expression | | Expression |
|---|---|---|---|---|
| Host | Phenotype | plasmid | phytase gene | level |
| KM71 | Mut^{s} | pPIC9K-WT | wt | 1.0 |
| KM71 | Mut^{s} | pPICNoT- | synthetic | 2.0 |
| | | G01651 | | |

### Example 7

### Constitutive Expression of Synthetic Citrobacter braakii Phytase Gene in Pichia pastoris

### Vectors:

pGAPZαA, commercial Pichia pastoris expression vector under GAP promoter for constitutive expression. Available from Invitrogen, Cat. No. 43-4500.

pPICNoT-G01651, expression construct containing synthetic cb phytase gene as described above.

### PCR Primers

| Oligo Name | Oligo Seq |
|---|---|
| OPhyg-Na | CGAAACCATGAGATTCCCATCCATCTTCACTG |
| | (SEQ ID NO: 44) |
| OPhyg-Nb | AAACCATGAGATTCCCATCCATCTTCACTG |
| | (SEQ ID NO: 45) |
| OAPhy-R | CATTCTGTTCCTCTCTCTTTTCCAAGGAAACACCTTC |
| | (SEQ ID NO: 46) |
| OAPhy-F | ggaaaagagaGAGGAACAGAATGGAATGAAGTTGG |
| | (SEQ ID NO: 47) |
| OPhy-Ca | AATTCTTACTCGGTGACAGCGCACTC |
| | (SEQ ID NO: 48) |
| OPhy-Cb II | CTTACTCGGTGACAGCGCACTC |
| | (SEQ ID NO: 49) |

### Host Strains:

Pichia pastoris GS115 (Mut⁺His⁻) (Invitrogen)

### Media:

YPD (1% yeast extract, 2% peptone, 2% D-glucose)

Phytase Activity Plate Assay:

15 μl of culture broth was applied into a 4 mm hole punched in the 1% agarose plate containing 0.2% phytic acid dodecasodium salt in 0.1M sodium acetate (pH5.5), The plate was incubated at 37° C. for 1 hr. 0.1M CaCl₂ in 0.2M sodium acetate (pH5.5) was overlaid on the plate for 30-60 min. The phytase activity was identified as a clear zone.

Phytase standard: Bio-feed phytase, batch 84-11401, 5191 FYT(V)/g

88.2 mg of Bio-feed phytase was dissolved into 104 ml of 0.1M sodium acetate (pH5.5) to prepare stock solution of the standard (4.4 FYT(V)/ml).

Construction of expression vectors pGAPα-G01651 using RIC cloning strategy:

Using plasmid pPICNoT-G01651 as the template, fragment 1 and 2 were amplified with primer paires OPhyg-Na/OPhy-Cb II and OPhyg-Nb/OPhy-Ca. The two fragments were purified by gel extraction kit and then annealed through annealing program. The annealed fragment was then subcloned into pGAPZaA vector at BstBI and EcoRI sites. The resulting expression construct pGAPα-G01651 was sequence confirmed.

Yeast Transformation of pGAPα-G01651—Synthetic Phytase:

P. pastoris GS115 was transformed using electroporation protocol, according to the Invitrogen manual. Competent cells were prepared as described and stored in 40 μl aliquots at −70° C. 5 μg of plasmid DNA was linearized with AvrII leading to insertion of the plasmid at the chromosomal 5′GAP locus. Linearized plasmid DNA (500 ng) was mixed with 40 μl of competent cells and stored on ice for 5 min. Cells were transferred to an ice-cold 0.2 cm electroporation cuvette. Transformation was performed using a BioRad GenePulser II. Parameters used were 1500 V, 25 μF and 200Ω Immediately after pulsing, cells were suspended in 1 ml of ice cold 1 M sorbitol, and incubated at 30° C. without shaking for 2 hrs. The mixtures were plated on YPDS plates containing 0.1, 1 mg/ml Zeocin, respectively. Plates were incubated at 28° C. for 3-4 days. Only a few colonies grew on 0.1 mg/ml Zeocin containing YPDS plate. Zeocin-resistant transformants were re-streaked on YPDS plates containing 0.1 mg/ml Zeocin and grown for 2 days before expression screening.

Expression Screening of Synthetic Phytase in P. pastoris Strain GS115 Under GAP Promoter in a 3 ml Scale:

67 candidate clones were tested for the expression of the desired protein. Screening was done in a 3 ml scale using 24-deep well plates (Whatman, UK). Cells were grown in YPD media over-night at 28° C. with vigorous shaking. Then the culture was diluted to 0.20D₆₀₀, and continuously grown for 4 days under the same growth condition. Samples of medium culture were taken daily, and stored at −20° C. for SDS-PAGE analysis and phytase activity assay.

The culture supernatant was analyzed by phytase activity plate assay. Strain A-G01651-94 (Mut^{s}, GS115) which expressed phytase under AOX1 promoter was used as a positive control. As a result, 62 out of 67 transformants had phytase activity. It appears that the phytase activity of transformants was similar to that of positive control strain at day 1. The activity was in-creased when the culture continued, but the fold of increase was less than the positive control. The bioactive samples of 24 transformants were run on SDS-PAGE gel for estimation of protein expression level. Clear band at expected size was observed in all transformants. With the incubation time increased, phytase protein accumulated, reaching the peak at day 3. Compared to strain A-G01651-94, the expression level of phytase from GAP construct is much lower.

### Screening of Multicopy Strains:

To select multi-copy recombinants, the strains were plated on increasing concentration of Zeocin. Two transformants with high copy of phytase genes were identified, namely, G-G01651-66 & G-G01651-67. Both strains could grow on 2 mg/ml Zeocin-containing plates. However, the expression of phytase in both strains was not significantly improved compared to other low copy strains.

TABLE 10
Expression data

| Strain | Signal Seq | Mature seq. | Expression in GS115 |
|---|---|---|---|
| G-G01651 | wt | Syn | low expression |

### Example 8

### Expression of Citrobacter braakii Phytase in Pichia methanolica

Media.

1.2M solbitol Scade plate (for 500 ml); 108 g of sorbitol fill up to 380 ml with water, 10 g of Agar Noble (Difco), after autoclave add the following solutions sterilized with 0.2 m filter, 50 ml of 10× basal salt w/o amino acid, 12.5 ml of 20% (w/v) casamino acid, 2 ml of 5% Threonin, 5 ml of 1% Triptophan, 50 ml of 20% glucose

10× basal salt w/o amino acid; 66.8 g/L of yeast nitrogen base w/o amino acid (Difco), 100 g/L of Succinic acid, 60 g/L of NaOH

YPD plate; 20 g/L of glucose, 20 g/L of peptone (Difco), 10 g/L of yeast extract (Difco) 20 g/L of agar

YPD; 20 g/L of glucose, 20 g/L of peptone (Difco), 10 g/L of yeast extract (Difco)

Pichia methanolica Expression System

Pichia methanolica expression was carried out using PMAD16 and pCZR134 (Yeast, 1998 vol14(1) p11).

### Primers:

(SEQ ID NO: 50)

Primer alpha-1;
5′-cgggaattcatgagattcccatccatcttc-3′

(SEQ ID NO: 51)

Primer alpha-2;
5′-cattccattctgttcctctctcttttccaaggaaac-3′

(SEQ ID NO: 52)

Primer phytase-3;
5′-ggaaaagagagaggaacagaatggaatgaag-3′

(SEQ ID NO: 53)

Primer phytase-4;
5′-gggactagtttactcggtgacagcgcactc-3′

### Construction of pCM and pCP

The phytase gene including α-factor signal sequence was designed based on the codon usage table of Pichia methanolica (www.kazusajp) and the rare codons in the gene were changed to the frequent codons of P. methanolica. The α-factor signal sequence from Saccharomyces cerevisiae (255 bp) was used. In addition 2 Step 13 cleavage sites (EAEA) were inserted between a-factor signal and mature sequence of phytase. The complete sequence is shown in SEQ ID NO: 7 (alpha-factor signal position 7-261; EAEA cleavage sites position 262-273; ORF of the C. braakii phytase from position 274-1509). The codon optimized gene was synthesized by DNA2.0. It was cloned in pJ2:G01847 with cloning restriction sites EcoRI and SpeI. The EcoRI-SpeI fragment of Citrobacter phytase gene from pJ2:G01847 was ligated into the EcoRI-SpeI site of pCZR134 then the expression vector pCM was constructed.

The Cirobacter baraakii phytase gene of which codon was optimized for P. pastoris (as in Example 5 and 6) was amplified by 2 steps of PCR. The 255 by of alpha-factor signal sequence gene was amplified using the PCR primers alpha-1 and alpha-2 with pPIC9K as template. The 1236 by of phytase gene was amplified with primer phytase-3 and phytase-4 with the pJ2:G01651 (Example 5 and 6) as template. A second PCR was carried out with the PCR products of phytase gene and alpha-factor signal gene with PCR primers alpha-1 and phytase-4. All PCR was carried out using Expand high fidelity polymerase (Roche) according to the product manual. The amplified phyatase gene with the alpha-factor signal sequence was ligated into EcoRI-SpeI site of pCRZ134 and the expression vector of pCP was constructed. The complete ORF codon optimized for P. pastoris including the alpha signal and the mature C. braakii phytase is shown in SEQ ID NO: 8.

Transformation of Pichia methanolica

The host strain, P. methanolica PMAD16, was transformed by electroporation using pCM and pCP. The transformants was isolated on 1.2M sorbitol SC plate and then they were cultivated onto YPD plate.

### Shaking Flask Cultivation

The transformants on YPD plates were inoculated to 50 ml of YPD liquid medium in 500 ml of a shaking flask and they were cultivated in a rotary shaker at 30 C for 24 hours. One ml of seed culture was inoculated to 50 ml of YPD liquid medium in 500 ml of a shaking flask and cultivated at 30 C. One ml of MeOH was added to the main culture on day 2 and day 3 and a sampling was carried out on day 3 and day 4.

TABLE 11
An overview of the results of expressing Citrobacter braakii
phytase in Pichia methanolica

| Expression plasmids | | | |
|---|---|---|---|
| transformed into | | codon optimization | |
| P. methanolica | Signal sequence | for | expression |
| pCM | Alpha factor | P. methanolica | Good |
| pCP | Alpha factor | P. pastoris | Very low |

### Example 9

### Comparative Expression of Three Different Synthetic Citrobacter braakii Phytase Genes in Aspergillus and Comparison Between Humicola insolens Cutinase Prepro Signal and Thermomyces lanuginosus Lipase Signal

Examples 1 to 3 describes expression of one particular synthetic gene sequence encoding C. braakii phytase. The codon optimization according to the present invention will however generate many synthetic gene sequences all encoding the same phytase. Below we have tested two additional synthetic genes encoding the C. braakii phytase and also compared different signal sequences.

### Primers:

SEQ ID NO: 54

P449
AGTCACCCTCTAGATCTCGAGCTACTCTGTGACGGCACACTCGGGC

SEQ ID NO: 55

P451
TATATACACAACTGGGGATCCCACCATGAAGTTCTTCACCACC

SEQ ID NO: 57

P456
TCGACGAATAGGACTGGCCAAG

SEQ ID NO: 58

P457
GGGGATCCACCATGAGGAGCTCCCTTGTG

SEQ ID NO: 59

P461
CTTGGCCAGTCCTATTCGTCGAGAGGAGCAGAACGGCATGAAATTG

SEQ ID NO: 60

P464
TTTTCTCGAGTCATTACTCGGTGAC

The plasmid pCOIs47 is a derivative of pJaL721 (Example 17, WO 03/008575), where a gene fragment of 1489 by has been inserted in the BamHI and XhoI sites as a stuffer for removal when inserting fragments in the BamHI and XhoI sites.

Construction of pCOIs514: A full length synthetic gene (position 67-1302 of SEQ ID NO: 2) en-coding the mature part of the C. braakii phytase including a cutinase-prepro signal was amplified by PCR from pCBPhycutiprepro (described in Example 3) using primers P449 (SEQ ID NO: 54) and P451 (SEQ ID NO: 55). The PCR product was cut with BamHI and XhoI and ligated in pCOIs47 cut with BamHI and XhoI. The insert was sequenced and verified to be identical to the original sequence.

Construction of pCOIs517: Another full length synthetic phytase gene (SEQ ID NO: 61, comprising the entire ORF encoding the mature phytase and including a Humicola insolens cutinase-prepro signal) was synthesized at DNA 2.0 (DNA 2.0 USA, 1430 O'Brian Drive, Suite E, Menlo Park, Calif. 94025 USA) and cut by BamHI and XhoI from a plasmid pCOIs536 delivered by DNA 2.0. The DNA fragment was ligated in pCOIs47 cut with BamHI and XhoI. The synthetic gene was designed as described in example 1.

Construction of pCOIs519: Another full length synthetic phytase gene (SEQ ID NO: 62, comprising the entire ORF encoding the mature phytase and including a Humicola insolens cutinase-prepro signal) was synthesized at DNA 2.0 (DNA 2.0 USA, 1430 O'Brian Drive, Suite E, Menlo Park, Calif. 94025 USA) and cut by BamHI and XhoI from a plasmid pCOIs536 delivered by DNA 2.0. The DNA fragment was ligated in pCOIs47 cut with BamHI and XhoI. The synthetic gene was designed as described in example 1.

Construction of pCOIs523: A nucleotide sequence (SEQ ID NO: 56) encoding the signal peptide from a lipase from Thermomyces lanuginosus (WO 97/04079) was fused to the synthetic gene encoding the mature part of the phytase (position 106 to 1341 of SEQ ID NO: 61) using SOE-PCR (splicing by overlap extension PCR). The PCR was performed using the primers P456 (SEQ ID NO: 57) and P457 (SEQ ID NO: 58) on Thermomyces lanuginosus lipae template and the primers P461 (SEQ ID NO: 59) and P464 (SEQ ID NO: 60) on pCOIs517 template. The PCR fragment was digested with BamHI and XhoI and ligated in pCOIs47 cut with BamHI and XhoI. The insert was sequenced and verified to be identical to the original sequence.

Cloning and Transformation in Aspergillus oryzae

The expression plasmids pCOIs514, pCOIs517, pCOIs519 and pCOIs523 were made as described above. The plasmids were transformed into Aspergillus oryzae BECh2 using amdS selection on plates containing acetamide as the sole nitrogen source. 30 transformants were isolated, grown in YPM for 3 days and supernatants run on an SDS-PAGE. This showed varying expression levels ranging from nothing to quite good expression—see table 12 for an expression summary. The predicted molecular weight is 46 kDa, however, the actual molecular weight is 60-70 kDa and highly glycosylated.

TABLE 12
An overview of expression in Aspergillus oryzae

| Expression plasmids transformed into | |
|---|---|
| Aspergillus | BECh2 (Aspergillus oryzae) |
| pCOIs514 | Very good expression |
| pCOIs517 | Very good expression |
| pCOIs519 | Very good expression |
| pCOIs523 | Very good expression |

1. A method for recombinant expression of a polypeptide from a gram negative bacterium in a fungal host cell, comprising the steps:
i) providing a nucleic acid sequence encoding the polypeptide, said nucleic acid sequence comprising a first nucleic acid sequence encoding a fungal signal peptide and a second nucleic acid sequence encoding the polypeptide, having at least one modified codon, wherein the modification does not change the amino acid encoded by said codon and the nucleic acid sequence of said codon is different compared to the corresponding codon in the wild type nucleic acid sequence present in the said gram negative bacterium;
ii) expressing the modified nucleic acid sequence in the fungal host.

2. The method according to claim 1, wherein at least 10% of the codons have been modified, particularly at least 20%, more particularly at least 30%, more particularly at least 50%, more particularly at least 75%, most particularly at least 90%.

3. The method according to claim 1, wherein the modification of at least one codon results in a codon optimized for translation in the fungal host organism.

4. The method according to claim 1, wherein the fungal host cell is a filamentous fungus or a yeast cell.

5. The method according to claim 4, wherein the filamentous fungal cell is selected from the group consisting of Acremonium, Aspergillus, Fusarium, Humicola, Mucor, Myceliophthora, Neurospora, Penicillium, Thielavia, Tolypocladium, or Trichoderma.

6. The method according to claim 4, wherein the yeast cell is Pichia.

7. The method according to claim 3, wherein codon usage of at least one modified codon corresponds to the codon usage of a fungal host cell selected from the group consisting of Acremonium, Aspergillus, Fusarium, Humicola, Mucor, Myceliophthora, Neurospora, Penicillium, Thielavia, Tolypocladium, Trichoderma or Pichia.

8. The method according to claim 3, wherein codon usage of at least one modified codon corresponds to the codon usage of a highly expressed gene in the fungal host cell.

9. The method according to claim 8, wherein codon usage of at least one modified codon corresponds to the codon usage of alpha amylase from Aspergillus oryzae.

10. The method according to claim 7, wherein the Aspergillus cell is Aspergillus awamori, Aspergillus foetidus, Aspergillus japonicus, Aspergillus niger, Aspergillus nidulans, or Aspergillus oryzae.

11. The method according to claim 6, wherein the Pichia cell is Pichia pastoris.

12. The method according to claim 1, wherein the gram negative bacterium is an Enterobacterium.

13. The method according to claim 12, wherein the Enterobacterium is selected from the group consisting of Escherichia sp and Citrobacter sp.

14. The method according to claim 13, wherein the Enterobacterium is selected from the group consisting of Escherichia coli, Citrobacter braakii, Citrobacter amalonaticus, Citrobacter gillenii.

15. The method according to claim 1, wherein the polypeptide is a hydrolase.

16. The method according to claim 15, wherein the hydrolase is a phytase or a phosphatase.

17. The method according to claim 16, wherein the modified nucleic acid sequence encoding the phytase is selected from the group consisting of SEQ ID NO: 2 from nucleotide position 67-1302, SEQ ID NO: 6 from nucleotide position 1-1236, and SEQ ID NO: 8 from nucleotide position 256-1491.

18. The method according to claim 1, wherein the nucleic acid sequence encoding the polypeptide further comprises a third nucleic acid sequence encoding a fungal propeptide, which third nucleic acid sequence is inserted between the first and the second nucleic acid sequences.

19. A fungal host cell comprising a DNA construct, said DNA construct comprising:
i) a first nucleic acid sequence encoding a fungal signal peptide; ii) a second nucleic acid sequence encoding a polypeptide from a gram negative bacterium; and wherein the second nucleic acid sequence comprises at least one modified codon compared to the wild type gene, which modification does not change the amino acid encoded by said codon.

20. A modified nucleic acid sequence encoding a phytase polypeptide and capable of expression in a fungal host organism, wherein said modified nucleic acid sequence differs in at least one codon from each wild type nucleic acid sequence encoding said phytase polypeptide.

21. The modified nucleic acid sequence according to claim 20, wherein at least 10% of the codons have been modified, particularly at least 20%, more particularly at least 30%, more particularly at least 50%, more particularly at least 75%, most particularly at least 90%.

22. The modified nucleic acid sequence according to claim 21, wherein the modification of at least one codon results in a codon optimized for translation in the fungal host organism.

23. The modified nucleic acid sequence according to claim 22, wherein codon usage of at least one modified codon corresponds to the codon usage of a fungal host cell selected from the group consisting of Acremonium, Aspergillus, Fusarium, Humicola, Mucor, Myceliophthora, Neurospora, Penicillium, Thielavia, Tolypocladium, Trichoderma or Pichia.

24. The modified nucleic acid sequence according to claim 23, wherein codon usage of at least one modified codon corresponds to the codon usage of alpha amylase from Aspergillus oryzae.

25. A modified nucleic acid sequence encoding a Citrobacter braakii phytase polypeptide and capable of expression in a fungal host organism, wherein:
a) the modified nucleic acid sequence has at least 80% identity with the nucleic acid sequence shown in SEQ ID NO: 2 position 67 to 1302; or
b) the modified nucleic acid sequence hybridizes under medium stringency conditions with the nucleic acid sequence shown in SEQ ID NO: 2 position 67 to 1302, or the complementary sequence thereof.

26. The modified nucleic acid sequence according to claim 25, consisting of the sequence shown in SEQ ID NO: 2 position 67 to 1302.

27-34. (canceled)


SEQUENCE LISTING (62 sequences)

SEQ ID NO: 1 - DNA, 1302 bp, Citrobacter braakii
atgagtacattcatcattcgtttattatttttttctctcttatgcggttctttctcaatacatgctgaagagcagaatggtatgaaacttgagcgggttgtgatagtgagtcgtcatggagtaagagcacctacgaagttcactccaataatgaaaaatgtcacacccgatcaatggccacaatgggatgtgccgttaggatggctaacgcctcgtgggggagaacttgtttctgaattaggtcagtatcaacgtttatggttcacgagcaaaggtctgttgaataatcaaacgtgcccatctccagggcaggttgctgttattgcagacacggatcaacgcacccgtaaaacgggtgaggcgtttctggctgggttagcaccaaaatgtcaaattcaagtgcattatcagaaggatgaagaaaaaaatgatcctctttttaatccggtaaaaatggggaaatgttcgtttaacacattgcaggttaaaaacgctattctggaacgggccggaggaaatattgaactgtatacccaacgctatcaatcttcatttcggaccctggaaaatgttttaaatttctcacaatcggagacatgtaagactacagaaaagtctacgaaatgcacattaccagaggctttaccgtctgaacttaaggtaactcctgacaatgtatcattacctggtgcctggagtctttcttccacgctgactgagatatttctgttgcaagaggcccagggaatgccacaggtagcctgggggcgtattacgggagaaaaagaatggagagatttgttaagtctgcataacgctcagtttgatcttttgcaaagaactccagaagttgcccgtagtagggccacaccattactcgatatgatagacactgcattattgacaaatggtacaacagaaaacaggtatggcataaaattacccgtatctctgttgtttattgctggtcatgataccaatcttgcaaatttaagcggggctttagatcttaactggtcgctacccggtcaacccgataatacccctcctggtggggagcttgtattcgaaaagtggaaaagaaccagtgataatacggattgggttcaggtttcatttgtttatcagacgctgagagatatgagggatatacaaccgttgtcgttagaaaaacctgctggcaaagttgatttaaaattaattgcatgtgaagagaaaaatagtcagggaatgtgttcgttaaaaagtttttccaggctcattaaggaaattcgcgtgccagagtgtgcagttacggaataa

SEQ ID NO: 2 - DNA, 1302 bp, Artificial Sequence
  C. braakii phytase codon optimized for expression in Aspergillus
atgtcgacattcatcattcgcttgttgttcttctcgttgttgtgtggatccttctccatccacgccgaagagcagaacggaatgaagttggagcgagtcgtgattgtctcgaggcacggagtccgcgcccctactaagttcacgcctatcatgaagaacgtcacccccgaccagtggcctcagtgggacgtgcctttgggatggctcacgccgcggggaggtgagctcgtctccgaactcggccagtaccagcgcctctggttcacatcgaaaggactcttgaacaaccagacttgtccttcgcccggacaggtcgcggtcattgcggataccgaccagcgcacaaggaagaccggtgaggcgttcctcgccggtttggcgcccaaatgtcagatccaggtccattaccagaaagacgaggagaaaaacgatcctttgttcaacccggtcaaaatgggcaaatgttcgttcaacactttgcaggtcaagaacgcaatcttggaacgcgcaggaggtaacattgagctctatacacagcgataccagtcgtcgttcaggaccctcgaaaacgtcttgaacttctcgcagtcggaaacatgtaagacgaccgagaagtcgactaaatgtaccctcccggaggcattgccttccgagttgaaggtcactcccgataacgtgtcgctccccggcgcgtggtcgttgtcgtcgacattgacggagatcttcctcctccaggaggcccagggcatgccccaggtcgcgtggggtaggatcaccggcgagaaggagtggagggacctcttgtccttgcataacgcacagttcgacttgttgcagcgcacccccgaagtggcaaggtcgagggcaactcccttgctcgatatgatcgatactgccctcttgaccaacggcaccaccgaaaaccggtacggtatcaaattgcccgtgtccctcttgttcatcgccggccacgataccaacttggcaaacctctccggcgccctcgatctcaactggtccctccctggtcagccggataacaccccgcctggcggagagctcgtcttcgagaaatggaagcggacgtcggataacacggactgggtccaggtctccttcgtctatcagaccttgagggatatgcgtgacatccagcccctctcgctcgagaagcccgccggtaaggtggacttgaaactcatcgcctgtgaggaaaagaactcgcagggtatgtgttcgctcaagtccttctcgcggctcattaaggagatccgtgtgcccgagtgtgccgtcacagagtaa

SEQ ID NO: 3 - DNA, 1311 bp, Citrobacter amalonaticus
atgaatacgctactttttcgattaataatgtttatattcatgtttggttctttcccattacaggcggaagtgccagatgacatgaagcttgaacgagttgtgatagtaagtcgccacggtgtaagagcaccaacaaagttcaccccattgatgcaggaaatcacaccttaccattggccgcaatgggatgttcccctgggctggttgacggctcggggtggtgagctcgtcaccgaaatgggacgatatcaacaaaaagtattaatcgataacggcgttctggaaagtaatgtatgtccgtcaccagaacaggtggcagttattgccgataccgatcagcgcactcgtaaaaccggtgaggcatttctggctggatttgcgccgggatgtaaaaataaggttcattatcaaaaagatcacgataaaaaagatcctctttttaatccagtaaaaatgggggtgtgcgcttttaatgtacaaaaaactcaggaagcgattctgacacgtgcggaaggaaacattgaacggtacactcagcgttatgactctgcattccgtactctggaacaggttctcaatttctcccggtcagcagcatgccgatcagcaagccagtctggttgcacgctaccaggaaccttaccttcagaactcagggtttctgcggataccgtttccttatctggcgcgtggagtctttcttccatgctgacggaaatatttctattgcaagaggcgcagggaatgccagaggttgcgtgggggcgaattcatggggagaaagaatggacagcgttattaagtctgcataatgctcagtttgaccttttgcaaagaactcccgaagttgcccgcagcagagcaacaccattactcgatttgatcagcgaagcattagtgagtaatgggtcaacagaaaatcattacggaattaaattacccgtctcattattgtttattgctggtcatgataccaatcttgcaaatctcagtggggtatttgatcttaactggtctctacctgggcagccagataatacacctcctggcggggagctggttttcgaaagatggacgcgagtgagtgataacactgactggattcaaatttcgtttgtttatcagactcttcaacaaatgcgtaagtttaaacctttttcatcttcgtctctcccaaacaagattgtgcttacgttgccctcttgccaggataaaaatcctgagggtatgtgtccattaaagcattttattgacattgtgcagacagcacgtattccacaatgtgcagtgatggctgatgtaaaccgttaa

SEQ ID NO: 4 - DNA, 1299 bp, Citrobacter gillenii
atgagtacactgatcattcgtttattgttcttaacgattatattggcccctgtttcattacgcgccgatgaacagagcggaatgcagcttgagcgtgttgtcatcgtcagtcgtcatggcgtcagggcaccgacaaagttcacgccgcttatgcagcaagtcactcccgaccgctggccgcaatgggacgttcctctggggtggttgactcctcgcggcggggcactcattactgagttaggacggtatcaacgtttacgcctggcggacaaaggtctgctggataataaaacgtgtccaacggcagggcaggtcgcggtcattgccgatagcgatcaacgtacccgtaaaacgggtgaagcattcctggcaggactggctccggaatgtaaagtacaggtttattatcaacaagataagtcaaaatctgatcccctttttaatcccatcaaggcggggcggtgttcgctgaacacatcgcaggtgaaagaggccatcctgacccgggctggcggaagtcttgatgagtacacgcgccactaccaacccgcatttcaagccctggaacgggtgttaaatttctcccagtcagaaaagtgtcaagcagctgggcagtctgcacagtgtacgctaaccgacgtcttacctgctgaactcaaggtctctccagaaaatatatcgttgtcaggctcatggggactggcttcaaccctgacggaaatcttcctgctgcaacaagcacaagggatgtcgcaggtggcctgggggcgtattcatggcgataaagaatggcgtacattattaagtctgcacaatgcgcagtttgaccttctgcagaaaaccccggaggttgcccgtagcagggccacaccgttacttgatttgatacgtacagcactcgtaacacagggggcaacagaaaataaatacgcaattcagttgcccgtctctttgttgtttattgcggggcatgacaccaatcttgccaatatcagcggggcattaggccttaacgtgtttctgcccggtcagccagataatacgccgccgggtggagagtttgttttcgaaaggtggaaacgggtcagcgatcattctgattgggtgcaggtttcttttatgtatcagacattgcaggaaatgcgtgatatgcaacctttgtcgttgcaatcgcctcccggaaaaattgtgctgcccttagcggcctgcgatgagaaaaatacgcagggaatgtgctcattaaaaaatttttctgcactgattgattccgttcgcgtgtccgaatgtgctgagaaataa

SEQ ID NO: 5 - DNA, 1236 bp, Citrobacter braakii
gaagagcagaatggtatgaaacttgagcgggttgtgatagtgagtcgtcatggagtaagagcacctacgaagttcactccaataatgaaaaatgtcacacccgatcaatggccacaatgggatgtgccgttaggatggctaacgcctcgtgggggagaacttgtttctgaattaggtcagtatcaacgtttatggttcacgagcaaaggtctgttgaataatcaaacgtgcccatctccagggcaggttgctgttattgcagacacggatcaacgcacccgtaaaacgggtgaggcgtttctggctgggttagcaccaaaatgtcaaattcaagtgcattatcagaaggatgaagaaaaaaatgatcctctttttaatccggtaaaaatggggaaatgttcgtttaacacattgcaggttaaaaacgctattctggaacgggccggaggaaatattgaactgtatacccaacgctatcaatcttcatttcggaccctggaaaatgttttaaatttctcacaatcggagacatgtaagactacagaaaagtctacgaaatgcacattaccagaggctttaccgtctgaacttaaggtaactcctgacaatgtatcattacctggtgcctggagtctttcttccacgctgactgagatatttctgttgcaagaggcccagggaatgccacaggtagcctgggggcgtattacgggagaaaaagaatggagagatttgttaagtctgcataacgctcagtttgatcttttgcaaagaactccagaagttgcccgtagtagggccacaccattactcgatatgatagacactgcattattgacaaatggtacaacagaaaacaggtatggcataaaattacccgtatctctgttgtttattgctggtcatgataccaatcttgcaaatttaagcggggctttagatcttaactggtcgctacccggtcaacccgataatacccctcctggtggggagcttgtattcgaaaagtggaaaagaaccagtgataatacggattgggttcaggtttcatttgtttatcagacgctgagagatatgagggatatacaaccgttgtcgttagaaaaacctgctggcaaagttgatttaaaattaattgcatgtgaagagaaaaatagtcagggaatgtgttcgttaaaaagtttttccaggctcattaaggaaattcgcgtgccagagtgtgcagttacggaataa

SEQ ID NO: 6 - DNA, 1236 bp, Artificial Sequence
  Citrobacter braakii phytase codon optimized for expression in P. pastoris
gaggaacagaatggaatgaagttggagagagttgtcatcgtttctagacacggtgttagagctcccaccaaattcactccaatcatgaagaacgtcaccccagatcagtggccacaatgggacgtcccactgggctggttgactccacgtggtggagaacttgtctctgaattgggtcagtaccagagactgtggttcacctccaaaggacttctgaataaccaaacttgcccatccccaggacaagtcgctgttattgccgacaccgatcaaagaaccagaaaaaccggagaggcctttttggcaggacttgctccaaaatgccagattcaagtccactaccaaaaagacgaagagaagaacgatccattgttcaatcccgtcaagatgggaaaatgctcctttaacaccttgcaagtcaaaaacgccattttggaaagagcaggtggcaatatcgagctttacacccagcgttaccaatcttcttttagaactttggaaaatgttttgaactttagtcagtccgagacttgcaagaccaccgagaagtctaccaagtgcactttgcccgaggctttgccctccgagcttaaggtcactcccgataacgtctccttgccaggagcatggtctctttcctccactttgaccgagattttcttgttgcaggaggcacaaggaatgccacaggtcgcatggggtagaattaccggtgaaaaggaatggagagacttgctgtctcttcacaacgcccagttcgatctcttgcagagaaccccagaggttgccagatccagagctactccacttttggatatgatcgacaccgctttgctgaccaatggtaccaccgagaacagatacggtattaagttgccagtctccttgctgttcattgcaggtcacgacaccaatttggccaacttgtctggagccttggacctgaactggtctttgccaggacagcccgacaataccccaccaggaggcgaattggttttcgaaaagtggaaaagaacctccgataacaccgattgggtccaagtctccttcgtctaccaaaccttgagagatatgcgtgacattcagccactgtctttggagaagcccgctggtaaggttgacttgaaattgatcgcttgcgaagaaaagaactcccagggaatgtgctctttgaagtccttttccagattgatcaaggagattagagtccccgagtgcgctgtcaccgagtaa

SEQ ID NO: 7 - DNA, 1515 bp, Artificial Sequence
  Citrobacter braakii phytase codon optimized for expression in P.methanolica
gaattcatgagattcccatccatcttcactgctgttttgttcgctgcttcctccgctttggctgctccagttaacactactactgaagatgaaactgctcaaatcccagctgaagctgttatcggttactccgacttagaaggtgatttcgacgttgctgttttgccattctccaactccaccaacaacggtttattgttcattaacaccaccattgcttccatcgctgctaaggaagaaggtgtttccttagaaaagagagaagctgaagctgaagaacaaaacggtatgaagttggaaagagttgttatcgtttccagacacggtgttagagctccaaccaagttcaccccaatcatgaagaacgtcaccccagaccaatggccacaatgggacgttccattgggttggttgaccccaagaggtggtgaattggtttccgaattgggtcaataccaaagattgtggttcacctccaagggcttgttgaacaaccaaacctgtccatcccctggtcaagtcgctgttatcgccgacaccgaccaaagaaccagaaagaccggtgaagctttcttggctggtttggctccaaagtgtcaaatccaagttcactaccaaaaggacgaagagaagaacgacccattgttcaacccagttaagatgggtaagtgttccttcaacaccttgcaagttaagaacgctatcttggaaagagccggtggtaacatcgaattatacacccaaagataccaatcctccttcagaaccttggaaaacgttttgaacttctcccaatccgaaacctgtaagaccaccgaaaagtccaccaagtgtaccttgccagaagctttgccatccgaattgaaggttaccccagacaacgtttccttgccaggtgcttggtccttgtcctccaccttgaccgaaatcttcttgttacaagaagctcaaggtatgccacaagttgcctggggtagaatcaccggtgaaaaggaatggagagacttgttgtccttgcacaacgcccaattcgacttgttgcaaagaaccccagaagttgctagatccagagctaccccattgttggacatgatcgacaccgccttgttgaccaacggcaccaccgaaaacagatacggtatcaagttgcctgtctccttgttgttcattgctggccacgacaccaacttagccaacttgtccggtgctttggacttaaactggtccttaccaggtcaaccagacaacaccccaccaggtggtgaattggttttcgaaaagtggaagagaacctccgacaacaccgactgggttcaagtttccttcgtttaccaaaccttgagagacatgagagacatccaaccattgtccttggaaaagccagctggtaaggtcgacttgaagttgatcgcttgtgaagaaaagaactcccaaggtatgtgttccttgaagtccttctccagattaatcaaggaaatcagagttcctgagtgtgctgttaccgaataaactagt

SEQ ID NO: 8 - DNA, 1491 bp, Artificial Sequence
  Citrobacter braakii phytase codon optimized for expression in P.pastoris
atgagattcccatccatcttcactgctgttttgttcgctgcttcctccgctttggctgctccagttaacactactactgaagatgaaactgctcaaatcccagctgaagctgttatcggttactccgacttggaaggtgatttcgacgttgctgttttgccattctccaactccaccaacaacggattgttgttcattaacaccaccattgcttccatcgctgctaaggaagaaggtgtttccttggaaaagagagaggaacagaatggaatgaagttggagagagttgtcatcgtttctagacacggtgttagagctcccaccaaattcactccaatcatgaagaacgtcaccccagatcagtggccacaatgggacgtcccactgggctggttgactccacgtggtggagaacttgtctctgaattgggtcagtaccagagactgtggttcacctccaaaggacttctgaataaccaaacttgcccatccccaggacaagtcgctgttattgccgacaccgatcaaagaaccagaaaaaccggagaggcctttttggcaggacttgctccaaaatgccagattcaagtccactaccaaaaagacgaagagaagaacgatccattgttcaatcccgtcaagatgggaaaatgctcctttaacaccttgcaagtcaaaaacgccattttggaaagagcaggtggcaatatcgagctttacacccagcgttaccaatcttcttttagaactttggaaaatgttttgaactttagtcagtccgagacttgcaagaccaccgagaagtctaccaagtgcactttgcccgaggctttgccctccgagcttaaggtcactcccgataacgtctccttgccaggagcatggtctctttcctccactttgaccgagattttcttgttgcaggaggcacaaggaatgccacaggtcgcatggggtagaattaccggtgaaaaggaatggagagacttgctgtctcttcacaacgcccagttcgatctcttgcagagaaccccagaggttgccagatccagagctactccacttttggatatgatcgacaccgctttgctgaccaatggtaccaccgagaacagatacggtattaagttgccagtctccttgctgttcattgcaggtcacgacaccaatttggccaacttgtctggagccttggacctgaactggtctttgccaggacagcccgacaataccccaccaggaggcgaattggttttcgaaaagtggaaaagaacctccgataacaccgattgggtccaagtctccttcgtctaccaaaccttgagagatatgcgtgacattcagccactgtctttggagaagcccgctggtaaggttgacttgaaattgatcgcttgcgaagaaaagaactcccagggaatgtgctctttgaagtccttttccagattgatcaaggagattagagtccccgagtgcgctgtcaccgagtaa

SEQ ID NO: 9 - DNA, 75 bp, Candida antartica
atgaagctactctctctgaccggtgtggctggtgtgcttgcgacttgcgttgcagccactcctttggtgaagtgc

SEQ ID NO: 10 - DNA, 75 bp, Candida albicans
atgaagctactctctctgaccggtgtggctggtgtgcttgcgacttgcgttgcagccactcctttggtgaagcgt

SEQ ID NO: 11 - DNA, 105 bp, Humicola insolens
atgaagttcttcaccaccatcctcagcaccgccagccttgttgctgctctccccgccgctgttgactcgaaccataccccggccgctcctgaacttgttgcccgg

SEQ ID NO: 12 - DNA, 255 bp, Saccharomyces cerevisiae
atgagatttccttcaattttcactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaga

SEQ ID NO: 13 - DNA, 255 bp, Artificial Sequence
  S. cerevisiae alpha factor signal codon optimized for expression in P. pastoris
atgagattcccatccatcttcactgctgttttgttcgctgcttcctccgctttggctgctccagttaacactactactgaagatgaaactgctcaaatcccagctgaagctgttatcggttactccgacttggaaggtgatttcgacgttgctgttttgccattctccaactccaccaacaacggattgttgttcattaacaccaccattgcttccatcgctgctaaggaagaaggtgtttccttggaaaagaga

SEQ ID NO: 14 - DNA, 37 bp, Artificial Sequence
  PCR primer
cctttggtgaagtgcgaagagcagaatggtatgaaac

SEQ ID NO: 15 - DNA, 37 bp, Artificial Sequence
  PCR primer
ccctctagatctcgagttattccgtaactgcacactc

SEQ ID NO: 16 - DNA, 39 bp, Artificial Sequence
  PCR primer
acacaactggggatccaccatgagtacattcatcattcg

SEQ ID NO: 17 - DNA, 36 bp, Artificial Sequence
  PCR primer
cctttggtgaagtgcgaagagcagaacggaatgaag

SEQ ID NO: 18 - DNA, 31 bp, Artificial Sequence
  PCR primer
agatctcgagaagcttactctgtgacggcac

SEQ ID NO: 19 - DNA, 37 bp, Artificial Sequence
  PCR primer
cctttggtgaagtgcgaagtgccagatgacatgaagc

SEQ ID NO: 20 - DNA, 38 bp, Artificial Sequence
  PCR primer
ccctctagatctcgagttaacggtttacatcagccatc

SEQ ID NO: 21 - DNA, 39 bp, Artificial Sequence
  PCR primer
acacaactggggatccaccatgaatacgctactttttcg

SEQ ID NO: 22 - DNA, 37 bp, Artificial Sequence
  PCR primer
cctttggtgaagtgcgatgaacagagcggaatgcagc

SEQ ID NO: 23 - DNA, 40 bp, Artificial Sequence
  PCR primer
ccctctagatctcgagttatttctcagcacattcggacac

SEQ ID NO: 24 - DNA, 39 bp, Artificial Sequence
  PCR primer
acacaactggggatccaccatgagtacactgatcattcg

SEQ ID NO: 25 - DNA, 40 bp, Artificial Sequence
  PCR primer
caactggggatctggtaccaccatgaagttcttcaccacc

SEQ ID NO: 26 - DNA, 41 bp, Artificial Sequence
  PCR primer
cttcattccgttctgctcttcggggagagcagcaacaaggc

SEQ ID NO: 27 - DNA, 41 bp, Artificial Sequence
  PCR primer
cttcattccgttctgctcttcccgggcaacaagttcaggag

SEQ ID NO: 28 - DNA, 21 bp, Artificial Sequence
  PCR primer
gaagagcagaacggaatgaag

SEQ ID NO: 29 - DNA, 49 bp, Artificial Sequence
  PCR primer
cagtcaccctctagatctcgacttaattaactactctgtgacggcacac

SEQ ID NO: 30 - DNA, 33 bp, Artificial Sequence
  PCR primer
gatccaaaccatgagatttccttcaattttcac

SEQ ID NO: 31 - DNA, 29 bp, Artificial Sequence
  PCR primer
caaaccatgagatttccttcaattttcac

SEQ ID NO: 32 - DNA, 34 bp, Artificial Sequence
  PCR primer
tctgctcttctcttttctcgagagataccccttc

SEQ ID NO: 33 - DNA, 37 bp, Artificial Sequence
  PCR primer
ctcgagaaaagagaagagcagaatggtatgaaacttg

SEQ ID NO: 34 - DNA, 29 bp, Artificial Sequence
  PCR primer
aattcttattccgtaactgcacactctgg

SEQ ID NO: 35 - DNA, 25 bp, Artificial Sequence
  PCR primer
cttattccgtaactgcacactctgg

SEQ ID NO: 36 - DNA, 17 bp, Artificial Sequence
  PCR primer
gatcctacgtagctgag

SEQ ID NO: 37 - DNA, 17 bp, Artificial Sequence
  PCR primer
aattctcagctacgtag

SEQ ID NO: 38 - DNA, 35 bp, Artificial Sequence
  PCR primer
gatccaaaccatgagattcccatccatcttcactg

SEQ ID NO: 39 - DNA, 31 bp, Artificial Sequence
  PCR primer
caaaccatgagattcccatccatcttcactg

SEQ ID NO: 40 - DNA, 37 bp, Artificial Sequence
  PCR primer
cattctgttcctctctcttttccaaggaaacaccttc

SEQ ID NO: 41 - DNA, 35 bp, Artificial Sequence
  PCR primer
ggaaaagagagaggaacagaatggaatgaagttgg

SEQ ID NO: 42 - DNA, 26 bp, Artificial Sequence
  PCR primer
aattcttactcggtgacagcgcactc

SEQ ID NO: 43 - DNA, 22 bp, Artificial Sequence
  PCR primer
cttactcggtgacagcgcactc

SEQ ID NO: 44 - DNA, 32 bp, Artificial Sequence
  PCR primer
cgaaaccatgagattcccatccatcttcactg

SEQ ID NO: 45 - DNA, 30 bp, Artificial Sequence
  PCR primer
aaaccatgagattcccatccatcttcactg

SEQ ID NO: 46 - DNA, 37 bp, Artificial Sequence; identical to SEQ ID NO: 40
  PCR primer

SEQ ID NO: 47 - DNA, 35 bp, Artificial Sequence; identical to SEQ ID NO: 41
  PCR primer

SEQ ID NO: 48 - DNA, 26 bp, Artificial Sequence
  PCR primer
aattcttactcggtgacagcgcactc

SEQ ID NO: 49 - DNA, 22 bp, Artificial Sequence
  PCR primer
cttactcggtgacagcgcactc

SEQ ID NO: 50 - DNA, 30 bp, Artificial Sequence
  PCR primer
cgggaattcatgagattcccatccatcttc

SEQ ID NO: 51 - DNA, 36 bp, Artificial Sequence
  PCR primer
cattccattctgttcctctctcttttccaaggaaac

SEQ ID NO: 52 - DNA, 31 bp, Artificial Sequence
  PCR primer
ggaaaagagagaggaacagaatggaatgaag

SEQ ID NO: 53 - DNA, 30 bp, Artificial Sequence
  PCR primer
gggactagtttactcggtgacagcgcactc

SEQ ID NO: 54 - DNA, 46 bp, Artificial Sequence
  PCR primer
agtcaccctctagatctcgagctactctgtgacggcacactcgggc

SEQ ID NO: 55 - DNA, 43 bp, Artificial Sequence
  PCR primer
tatatacacaactggggatcccaccatgaagttcttcaccacc

SEQ ID NO: 56 - DNA, 66 bp, Thermomyces lanuginosus
atgaggagctcccttgtgctgttctttgtctctgcgtggacggccttggccagtcctattcgtcga

SEQ ID NO: 57 - DNA, 22 bp, Artificial Sequence
  PCR primer
tcgacgaataggactggccaag

SEQ ID NO: 58 - DNA, 29 bp, Artificial Sequence
  PCR primer
ggggatccaccatgaggagctcccttgtg

SEQ ID NO: 59 - DNA, 46 bp, Artificial Sequence
  PCR primer
cttggccagtcctattcgtcgagaggagcagaacggcatgaaattg

SEQ ID NO: 60 - DNA, 25 bp, Artificial Sequence
  PCR primer
ttttctcgagtcattactcggtgac

SEQ ID NO: 61 - DNA, 1341 bp, Artificial Sequence
  C. braakii phytase codon optimized for A. oryzae with cutinase signal peptide
atgaagttcttcaccaccatcctcagcaccgccagccttgttgctgctctccccgccgctgttgactcgaaccataccccggccgctcctgaacttgttgcccgggaggagcagaacggcatgaaattggagcgggtggtgattgtatcgaggcacggagttagggcacccaccaagttcacccccatcatgaaaaacgtcacacccgaccagtggcctcaatgggacgtcccgctcggctggctcactccccgtggaggcgaactcgtctcggagcttggtcagtaccagaggctctggttcacgtctaagggtctcttgaacaaccagacatgtccgagtccaggccaagtcgccgttattgccgacacagatcagaggacccgaaagactggtgaagccttcctcgcaggactggctccaaagtgtcagattcaagtacactaccagaaggacgaggagaagaacgacccgctcttcaacccggtcaagatgggcaaatgtagcttcaacaccctccaggtgaaaaacgccatccttgaacgagccggtggcaacattgagctctacacacagaggtaccaatctagcttccggacgctggagaacgtcttgaacttttcgcagtcggagacttgtaaaaccacggagaagtcgacaaaatgtactctgccggaggccttgccgagtgaactcaaggtcacacccgacaacgtttcccttccgggtgcatggtcactctcctcaaccttgacggagatcttccttttgcaggaagcgcaggggatgccccaggtggcctggggacgtatcactggcgagaaggagtggagggacctcctctccctccacaacgcgcagtttgacctcctccaacgcacaccggaagtcgcaaggtcgcgtgccacaccgttgcttgacatgatcgacacagcattgctcacgaacggaactacggaaaaccgctatggcatcaaactccctgtgtccctcctcttcattgctggccacgacaccaacctcgcaaacctttccggtgctctggatctcaactggtcgcttcctggccagccggacaacactcctccgggtggggagctggtcttcgaaaaatggaagaggacaagcgataacacggattgggttcaggttagtttcgtataccagactttgcgcgacatgcgggatatccaaccgctttctctggaaaaacctgcagggaaggtggatctcaagctcatcgcgtgtgaggagaaaaacagccagggcatgtgttcattgaagtcattctcacgcttgattaaggagattcgggtcccggagtgtgcggtcaccgagtaa

SEQ ID NO: 62 - DNA, 1341 bp, Artificial Sequence
  C. braakii phytase codon optimized for A. oryzae with cutinase signal peptide
atgaagttcttcaccaccatcctcagcaccgccagccttgttgctgctctccccgccgctgttgactcgaaccataccccggccgctcctgaacttgttgcccgggaggagcagaacggcatgaagttggagagggtcgtcatcgtgtcgaggcacggcgtcagggcccccaccaagttcacccccatcatgaagaacgtcacccccgaccagtggccccagtgggacgtccccctcggctggctcacccccaggggcggcgagctcgtctcggagctcggccagtaccagaggctctggttcacctcgaagggcctcctcaacaaccagacctgtccctcgcccggccaggtcgccgtcatcgccgacaccgaccagaggaccaggaagaccggcgaggccttcctcgccggcctcgcccccaagtgtcagatccaggtccactaccagaaggacgaggagaagaacgaccccctcttcaaccccgtcaagatgggcaagtgttcgttcaacaccctccaggtcaagaacgccatcttggagagggccggcggcaacatcgagctctacacccagaggtaccagtcgtcgttcaggaccttggagaacgtcctcaacttctcgcagtcggagacctgtaagaccaccgagaagtcgaccaagtgtaccctccccgaggccctcccctcggagctcaaggtcacccccgacaacgtctcgctccccggcgcctggtcgctctcgtcgaccctcaccgagatcttcctcctccaggaggcccagggcatgccccaggtcgcctggggcaggatcaccggcgagaaggagtggagggacctcctctcgctccacaacgcccagttcgacctcctccagaggacccccgaggtcgccaggtcgagggccacccccctcctcgacatgatcgacaccgccctcctcaccaacggcaccaccgagaacaggtacggcatcaagctccccgtctcgctcctcttcatcgccggccacgacaccaacctcgccaacctctcgggcgccctcgacctcaactggtcgctccccggccagcccgacaacaccccccccggcggcgagctcgtcttcgagaagtggaagaggacctcggacaacaccgactgggtccaggtctcgttcgtctaccagaccctcagggacatgagggacatccagcccctctcgttggagaagcccgccggcaaggtcgacctcaagctcatcgcctgtgaggagaagaactcgcagggcatgtgttcgctcaagtcgttctccaggctcatcaaggagatcagggtccccgagtgtgccgtcaccgagtaa